(19) United States
(12) Patent Application Publication
(10) Pub. No.: US 2003/0119894 A1
(43) Pub. Date: Jun. 26, 2003

(54) Methods for treatment of cancer or neoplastic disease and for inhibiting growth of cancer cells and neoplastic cells

(75) Inventors: Madiraju S.R. Murthy, Brossard (CA); Gordon C. Shore, Montreal (CA); Jurgen Bajorath, Lynnwood, WA (US); Florence L. Stahura, Sammamish, WA (US)

Correspondence Address: PENNIE AND EDMONDS, 1155 AVENUE OF THE AMERICAS, NEW YORK, NY, 100362711

(73) Assignee: Gemin X Biotechnologies Inc.

(21) Appl. No.: 09/910,291

(22) Filed: Jul. 20, 2001

(51) Int. Cl. A61K 31/404

(52) U.S. Cl. 514/414

(57) ABSTRACT

The present invention provides methods for treating or preventing cancer or neoplastic disease comprising administering to a patient a compound having the features of a pharmacophore for human anti-apotptotic Bcl protein inhibitors or identified by the in vitro methods for identifying anti-apotptotic-Bcl protein inhibitors. Also disclosed are methods for inhibiting the growth of a cancer cell or a neoplastic cell, comprising contacting the cancer cell or neoplastic cell with a compound having the features of a pharmacophore for human anti-apoptotic-Bcl protein inhibitors.

### 1. FIELD OF THE INVENTION

The present invention relates to methods for treating or preventing cancer or neoplastic disease in a patient, comprising administering to a patient a compound having the features of a pharmacophore as defined herein. The methods of the present invention are also useful for inhibiting the growth of a cancer cell or a neoplastic cell.

### 2. BACKGROUND OF THE INVENTION

Cancer affects approximately 20 million adults and children worldwide, and this year, more than 9 million new cases will be diagnosed (International Agency for Research on Cancer; www.irac.fr). According to the American Cancer Society, about 563,100 Americans are expected to die of cancer this year, more than 1500 people a day. Since 1990, in the United States alone, nearly five million lives have been lost to cancer, and approximately 12 million new cases have been diagnosed.

Currently, cancer therapy involves surgery, chemotherapy and/or radiation treatment to eradicate neoplastic cells in a patient (see, for example, Stockdale, 1998, “Principles of Cancer Patient Management,” in Scientific American: Medicine, vol. 3, Rubenstein and Federman, eds., Chapter 12, Section IV). All of these approaches pose significant drawbacks for the patient. Surgery, for example, may be contraindicated due to the health of the patient or may be unacceptable to the patient. Additionally, surgery may not completely remove the neoplastic tissue. Radiation therapy is effective only when the irradiated neoplastic tissue exhibits a higher sensitivity to radiation than normal tissue, and radiation therapy can also often elicit serious side effects. (Id.) With respect to chemotherapy, there are a variety of chemotherapeutic agents available for treatment of neoplastic disease. However, despite the availability of a variety of chemotherapeutic agents, chemotherapy has many drawbacks (see, for example, Stockdale, 1998, “Principles Of Cancer Patient Management” in Scientific American Medicine, vol. 3, Rubenstein and Federman, eds., ch. 12, sect. 10). Almost all chemotherapeutic agents are toxic, and chemotherapy causes significant, and often dangerous, side effects, including severe nausea, bone marrow depression, immunosuppression, etc. Additionally, many tumor cells are resistant or develop resistance to chemotherapeutic agents through multi-drug resistance.

Tamura et al., JP93086374, discloses metacycloprodigiosin and/or prodigiosin-25C as being useful for treating leukemia, but provides data for only prodigiosin-25C activity against L-5178Y cells in vitro. Hirata et al., JP-10120562, discloses the use of cycloprodigiosin as an inhibitor of the vacuolar ATPase proton pump and states that cycloprodigiosin may have anti-tumor enhancing activity. Hirata et al., JP-10120563 discloses the use of cycloprodigiosin as a therapeutic drug for leukemia, as an immunosuppressant, and as an apoptosis inducer. JP61034403, to Kirin Brewery Co. Ltd, describes prodigiosin for increasing the survival time of mice with leukemia. Boger, 1988, J. Org. Chem. 53:1405-1415 discloses in vitro cytotoxic activity of prodigiosin, prodigiosene, and 2-methyl-3-pentylprodigiosene against mouse P388 leukemia cells. The National Cancer Institute, http://dtp.nci.nih.gov, discloses data obtained from the results of a human-tumor-cell-line screen, including screening of butylcycloheptyl-prodiginine HCl; however, the screen provides no indication that the compounds of the screen are selective for cancer cells (e.g., as compared to normal cells).

In multicellular organisms, elimination of certain individual cells in an organized and programmed fashion is part of the developmental process. Such a process of elimination of cells is known as programmed cell death, or apoptosis. Cells undergo apoptosis once they fulfill their role in tissue development, when they are infected with viruses, or when normal growth is compromised due to genetic anomalies that can lead to cancer. Thus, apoptosis is a defense mechanism by which only affected cells are eliminated and the organism is spared. Cancerous growth of cells results when aberrant cells bypass the apoptosis pathway, either by inactivation of genes that promote apoptosis or by activation of cell-death inhibitors (see e.g. Hanahan et al. 2000 Cell 100: 57-70).

In many cells, a signal for the expression of a transforming oncogene also leads to apoptosis (Hoffman et al. (1998) Oncogene 17: 3351-58). However, in other instances, the same signal can lead to uncontrolled cell proliferation and cancer where survival of those cells is controlled by the “survival/death set point” of the cell. In most cells, the survival/death set point is regulated by interactions between and anti-apoptotic and pro-apoptotic proteins. Proteins included within the Bcl family of polypeptides include both anti-apoptotic proteins and pro-apoptotic proteins, which either interfere with or facilitate apoptosis, respectively. Members of the anti-apoptotoic family of Bcl proteins include, but are not limited to Bcl-2, Bcl-w, Mcl-1, Bcl-x1 and their homologues, while members of the pro-apoptotic family of Bcl proteins include, but are not limited to, Bax, Bad, Bid, Bak and their homologues. Regulation of the set point is controlled by the activities of these proteins, which are in turn controlled through hetero- and homodimerization (Reed (1998) Oncogene 17:3225-3236). Cells become resistant to death signals when anti-apoptotic Bcl protein are present in relative excess, such that the equilibrium is shifted toward formation of anti-apoptotic Bcl protein homodimers and anti-apoptotic Bcl protein/pro-apoptotic Bcl protein heterodimers. Conversely, when anti-apoptotic Bcl protein levels are low, homodimers of pro-apoptotic Bcl protein predominate, resulting in cell susceptibility to death signals. For example, Bad regulates the Bcl-2/Bax equilibrium by competing with Bax for binding to Bcl-2, thereby promoting the formation of Bax homodimers and cell death. Bad does not interact with Bax, and thus has no direct effect on levels of Bax homodimers. Thus, in this example, compounds that bind to Bcl-2 and disrupt Bcl-2 homodimer and Bcl-2/Bax heterodimer formation promote apoptosis in cells, particularly cancerous and neoplastic cells, that receive a death signal but would otherwise be resistant to death as a result of the presence of high levels of Bcl-2.

In this illustrative example, Bcl-2 and its homologues are present on the outer mitochondrial membrane, endoplasmic reticulum and nuclear envelope of cells and are believed to counteract cell death at various locations. Bax is likely to exert its death-promoting effects by acting on the mitochondrial outer membrane, resulting in the release of cytochrome C. In turn, cytochrome C that is released from mitochondria participates in the conversion and resultant activation of procaspase-9 to caspase-9, one of the initiator caspases. Caspases are proteases involved in the cell death pathway that ultimately activate DNA-degrading enzymes in the nucleus and lead to chromosomal breakdown. Bcl-2 inhibits the release of cytochrome C and antagonizes the cell death pathway, most likely by interacting with Bax and preventing Bax homodimer formation. If the survival/death set point of a cell is biased towards survival by increasing the expression of Bcl-2 protein, then when that cell receives a death signal triggered, e.g., by expression of an oncogene or by exposure to drug therapies or radiation, it will escape apoptosis and proliferate, which can lead to cancer or neoplastic disease.

Therefore, compounds that bind to Bcl-2 and readjust the set point of neoplastic cells or cancer cells toward cell death can be effective anti-cancer drugs. Such compounds include peptides derived from the Bax or Bad regions that participate in interactions with Bcl-2 in vivo. Only a few anti-apoptotic Bcl protein-binding, and more specifically, Bcl-2-binding compounds capable of inhibiting the Bcl-2/Bax interaction are currently available (see e.g., Wang et al. 2000, Proc. Natl. Acad. Sci. USA 97 (13): 7124-29). Moreover, these compounds are 13-amino-acid peptides from the BH₁ and BH₃ domains of Bad, which not only are susceptible to proteolytic degradation but they also have poor bioavailability across cell membranes.

Accordingly, there is a need for anti-apoptotic Bcl protein-binding compounds that are resistant to degradation, have good bioavailability and disrupt anti-apoptotic Bcl protein/pro-apoptotic Bcl protein interactions in order to promote death of neoplastic and cancer cells.

Citation or identification of any reference in Section 2 of this application is not be construed as an admission that such reference is prior art to the present application.

### 3. SUMMARY OF THE INVENTION

The present invention also relates to a method for treating or preventing cancer or eoplastic disease in a patient, comprising administering to a patient in need thereof an effective amount of a compound or a pharmaceutically acceptable salt thereof having the features of a two-dimensional pharmacophore. In this embodiment of the invention, the pharmacophore has a first heterocyclic aromatic ring (Ring A), a second heterocyclic aromatic ring (Ring B) substituted with a polar group, a third heterocyclic aromatic ring (Ring C), and an aliphatic group, and each aromatic ring and the aliphatic group has a centroid, and the centroids are separated from other centroids by the distances indicated in FIG. 1A and Table 2.

In another embodiment, the invention relates to a method for treating or preventing cancer or neoplastic disease in a patient, comprising administering to a patient in need thereof an effective amount of a compound or a pharmaceutically acceptable salt thereof having the features of a three-feature, three-dimensional pharmacophore. In this embodiment, the pharmacophore has a hydrogen bond acceptor feature (A1), a first hydrogen bond donor feature (D1) and a second hydrogen bond donor feature (D2), in which D1, A,1 and D2 each has a centroid, and where each centroid is separated from the other centroids by the following distances:

| Pair of features | Distance between the features |
|---|---|
| A1-D1 | 2.5-4.5 Å |
| A1-D2 | 2.5-4.5 Å |
| D1-D2 | 3.5-5.5 Å. |

The present invention is further directed to a method of treating or preventing cancer or neoplastic disease in a patient, comprising administering to a patient in need thereof an effective amount of a compound having the features of another three-feature, three-dimensional pharmacophore. In this embodiment of the invention, the pharmacophore has a hydrogen bond acceptor feature (A1), a polar group feature (P1), and a hydrogen bond donor feature (D1) in which A1, D1 and P1 each has a centroid, where each centroid separated from the other centroids by the following distances:

| Pair of features | Distance between the features |
|---|---|
| A1-D1 | 2.5-4.5 Å |
| P1-D1 | 4.5-6.5 Å |
| P1-A1 | 2.5-4.5 Å |

The present invention is still further directed to a method of treating or preventing cancer or neoplastic disease in a patient, comprising administering to a patient in need thereof an effective amount of a compound having the features of a four-point three-dimensional pharmacophore. In this embodiment of the invention, the pharmacophore has a first hydrogen bond donor feature (D1), a hydrogen bond acceptor feature (A1), a second hydrogen bond donor feature (D2), and a polar group feature (P1), in which D1, A1, D2, and P1, each has a centroid, where each centroid separated from the other centroids by the following distances:

| Pair of features | Distance between the features |
|---|---|
| A1-D1 | 2.5-4.5 Å |
| A1-D2 | 2.5-4.5 Å |
| D1-D2 | 3.5-5.5 Å |
| P1-D1 | 4.5-6.5 Å |
| P1-A1 | 2.5-4.5 Å |
| P1-D2 | 4.5-6.5 Å. |

The present invention is also directed toward a method for treating or preventing cancer or neoplastic disease in a patient, comprising administering to a patient in need thereof an effective amount of a compound of Formula III:

A—B—X—C (III)

or a pharmaceutically acceptable salt thereof, where A is selected from the group consisting of

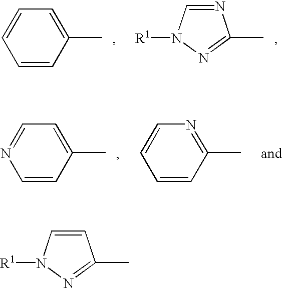

and optionally substituted at one or more carbon atoms with one or more —C₁-C₆, —OC₁-C₆, —OC(O)C₁-C₆, —C(O)C₁-C₆, —C(O)OC₁-C₆, —CF₃, —NO₂, —CH₂O—C₁-C₆, or halo roups, R¹ is selected from the group consisting of H, —C₁-C₆ and —C(O)C₁-C₆; and B is elected from the group consisting of:

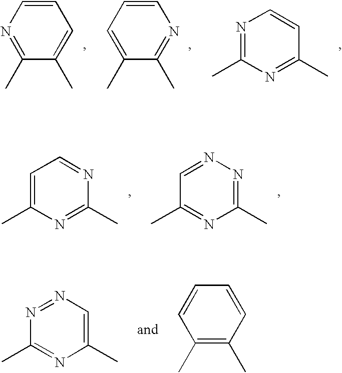

and optionally substituted at one or more carbon atoms with one or more —C₁-C₆, —OC₁-C₆, OC(O)C₁-C₆, —C(O)C₁-C₆, —C(O)OC₁-C₆, —CF₃, —NO₂, —CH₂O—C₁-C₆, or halo groups, X is selected from the group consisting of —O—, —S— and —N(H)—; and C is selected from the group consisting of

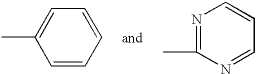

and optionally substituted at one or more carbon atoms with one or more —C₁-C₆, —OC₁-C₆, —OC(O)C₁-C₆, —C(O)C₁-C₆, 13 C(O)OC₁-C₆, —CF₃, —NO₂, —CH₂O—C₁-C₆, or halo groups.

The present invention also relates to a method for inhibiting the growth of a cancer cell or neoplastic cell, comprising contacting the cancer cell or neoplastic cell with an effective amount of a compound or a pharmaceutically acceptable salt thereof having the features of a two-dimensional pharmacophore. In this embodiment of the invention, the pharmacophore has a first heterocyclic aromatic ring (Ring A), a second heterocyclic aromatic ring (Ring B) substituted with a polar group, a third heterocyclic aromatic ring (Ring C), and an aliphatic group, and each aromatic ring and the aliphatic group has a centroid, and the centroids are separated from other centroids by the distances indicated in FIG. 1A and Table 2.

In another embodiment, the present invention relates to a method for inhibiting the growth of a cancer cell or neoplastic cell, comprising contacting the cancer cell or neoplastic cell with an effective amount of a compound or a pharmaceutically acceptable salt thereof having the features of a three-feature, three-dimensional pharmacophore. In this embodiment, the pharmacophore has a hydrogen bond acceptor feature (A1), a first hydrogen bond donor feature (D1), and a second hydrogen bond donor feature (D2), in which D1, A1 and D2 each has a centroid, and where each centroid is separated from the other centroids by the following distances:

| Pair of features | Distance between the features |
|---|---|
| A1-D1 | 2.5-4.5 Å |
| A1-D2 | 2.5-4.5 Å |
| D1-D2 | 3.5-5.5 Å. |

The present invention also relates to a method for inhibiting the growth of a cancer cell or neoplastic cell, comprising contacting the cancer cell or neoplastic cell with an effective amount of a compound or a pharmaceutically acceptable salt thereof having the features of another three-feature, three-dimensional pharmacophore. In this embodiment, the pharmacophore has a hydrogen bond acceptor feature (A1), a polar group feature (P1), and a hydrogen bond donor feature (D1), in which D1, A1, and P1, each has a centroid, where each centroid separated from the other centroids by the following distances:

| Pair of features | Distance between the features |
|---|---|
| A1-D1 | 2.5-4.5 Å |
| P1-D1 | 4.5-6.5 Å |
| P1-A1 | 2.5-4.5 Å |

The present invention is also directed toward a method for inhibiting the growth of a cancer cell or a neoplastic cell, comprising contacting the cancer cell or neoplastic cell with an effective amount of a compound having the features of a four-feature, three-dimensional pharmacophore. In this embodiment of the invention, the pharmacophore has a first hydrogen bond donor feature (D1), a hydrogen bond acceptor feature (A1), a second hydrogen bond donor feature (D2), and a polar group feature (P1), in which D1, A1, D2, and P1, each has a centroid, where each centroid separated from the other centroids by the following distances:

| Pair of features | Distance between the features |
|---|---|
| A1-D1 | 2.5-4.5 Å |
| A1-D2 | 2.5-4.5 Å |
| D1-D2 | 3.5-5.5 Å |
| P1-D1 | 4.5-6.5 Å |
| P1-A1 | 2.5-4.5 Å |
| P1-D2 | 4.5-6.5 Å. |

The present invention is also directed toward a method for inhibiting the growth of a cancer cell or a neoplastic cell, comprising contacting the cancer cell or neoplastic cell with an effective amount of a compound of Formula III:

A—B—X—C (III)

or a pharmaceutically acceptable salt thereof, where A is selected from the group consisting of

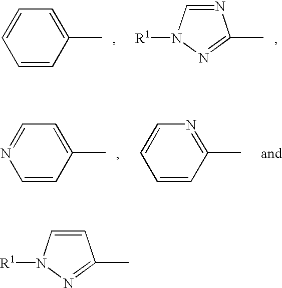

and optionally substituted at one or more carbon atoms with one or more —C₁-C₆, —OC₁-C₆, —OC(O)C₁-C₆, —C(O)C₁-C₆, —C(O)OC₁-C₆, —CF₃, —NO₂, —CH₂O—C₁-C₆, or halo groups, R¹ is selected from the group consisting of H, —C₁-C₆ and —C(O)C₁-C₆; and B is selected from the group consisting of:

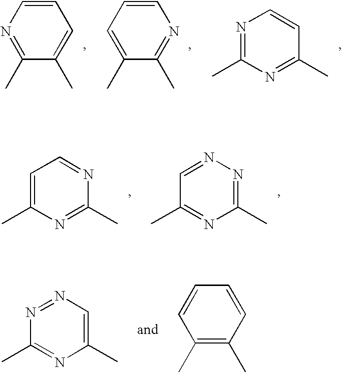

and optionally substituted at one or more carbon atoms with one or more —C₁-C₆, —OC₁-C₆, —OC(O)C₁-C₆, —C(O)C₁-C₆, —C(O)OC₁-C₆, —CF₃, —NO₂, —CH₂O—C₁-C₆, or halo groups, X is selected from the group consisting of —O—, —S— and —N(H)—; and C is selected from the group consisting of

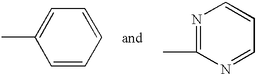

and optionally substituted at one or more carbon atoms with one or more —C₁-C₆, —OC₁-C₆, —OC(O)C₁-C₆, —C(O)C₁-C₆, —C(O)OC₁-C₆, —CF₃, —NO₂, —CH₂O—C₁-C₆, or halo groups.

The present invention may be understood more fully by reference to the figures, detailed description and examples, which are intended to exemplify non-limiting embodiments of the invention.

### 4. BRIEF DESCRIPTION OF THE DRAWINGS

FIG. 1 shows pharmacophores based on the prodigiosin chemotype: (A) A two-dimensional pharmacophore; (B) a three-feature pharmacophore superimposed on the structure of streptorubin B; (C) a four-feature pharmacophore superimposed on the structure of streptorubin B.

FIG. 2 depicts a computer system for selecting compounds of the present invention from a database of chemical compounds.

FIG. 3 shows the ability of compounds of the present invention to induce apoptosis, selectively, in different types of cancer cells.

FIG. 4 shows the effect of streptorubin B in reinstating apoptosis in cells that over-express Bcl-2.

FIG. 5 shows the effect of streptorubin B in inhibiting transformed cells (A), and in killing transformed cells (B).

### 5. DETAILED DESCRIPTION OF THE INVENTION

The present invention relates to methods for treating or preventing cancer or neoplastic disease in a patient, comprising administering to a patient in need of such treatment or prevention a compound selected as having the features of a pharmacophore disclosed herein. The compounds include anti-apoptotic Bcl protein-inhibitors, or have, in particular, the ability to inhibit Bcl-2 interactions with Bax. The inhibition of the illustrative Bcl-2/Bax interaction is measurable using the in vitro assays disclosed herein. The pharmacophore is based on chemotype molecules of the prodigiosin family, referred to herein as the “prodigiosin chemotype.” Compounds of the prodigiosin chemotype: (1) inhibit Bcl-2 homodimerization, (2) inhibit interactions between Bcl-2 and Bax, and (3) selectively promote cell death in Bcl-2-overproducing cancer or neoplastic cells.

The present invention also relates to methods for inhibiting the growth of a cancer cell or a neoplastic cell, comprising contacting the cancer cell or neoplastic cell with an effective amount of a compound having the features of a pharmacophore disclosed herein.

As used herein, a “database” of compounds contains one or more compounds to be screened using the products and methods of the present invention. Examples of such databases include, but are not limited to, the Cambridge Crystallographic Database (Cambridge Crystallographic Data Centre, Cambridge, U.K.), the ACD Database (MDL Information Systems, Inc., San Leandro, Calif.), and the Beilstein Database (Beilstein Chemiedaten und Software GmbH, Frankfurt, Del.).

The phrase “pharmaceutically acceptable salt(s),” as used herein includes but is not limited to salts of acidic or basic groups that may be present in compounds identified using the methods of the present invention. Compounds that are basic in nature are capable of forming a wide variety of salts with various inorganic and organic acids. The acids that can be used to prepare pharmaceutically acceptable acid addition salts of such basic compounds are those that form non-toxic acid addition salts, i.e., salts containing pharmacologically acceptable anions, including but not limited to sulfuric, citric, maleic, acetic, oxalic, hydrochloride, hydrobromide, hydroiodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds that include an amino moiety can form pharmaceutically or cosmetically acceptable salts with various amino acids, in addition to the acids mentioned above. Compounds that are acidic in nature are capable of forming base salts with various pharmacologically or cosmetically acceptable cations. Examples of such salts include alkali metal or alkaline earth metal salts and, particularly, calcium, magnesium, sodium, lithium, zinc, potassium, and iron salts.

The terms “heterocyclic aromatic group,” and “heterocyclic aromatic ring,” as used herein, refer to an aromatic ring having one or more nitrogen, oxygen or sulfur atoms. Heterocyclic aromatic groups include, but are not limited to, pyrrolyl, imidazolyl, 1,3,4-triazolyl, tetrazolyl, furanyl, thienyl, pyridyl, pyrrolyl, azepinyl, azirinyl, benzothiophenyl, benzotriazolyl, indazolyl, indolyl, isoquinolinyl, isothiazolyl, phenanthridinyl, phenazinyl, phthalazinyl, pteridinyl, purinyl, pyrazinyl, pyrazolyl, pyridazinyl, pyridinyl, pyrimidinyl, pyrrolyl, quinazolinyl, quinolinyl, quinoxalinyl, tetrazinyl, tetrazolyl, thiazolyl, thiophenyl, triazinyl, triazolyl groups, and pyrimidyl groups.

The term “aliphatic group,” as used herein, refers to a group having only carbon and hydrogen atoms. Aliphatic groups include, but are not limited to, C₁-C₁₂ straight or branched chain alkyl groups, C₂-C₁₂ straight or branched chain alkenyl groups, and C₂-C₁₂ straight or branched chain alkynyl groups.

The term “aromatic group,” as used herein, refers to an unsaturated cyclic or polycyclic ring system having a conjugated π electron system. Specifically included within the definition of “aromatic group” are phenyl, benzyl, naphthyl, anthracenyl, phenanthracenyl, benzanthracenyl, chrysenyl, and triphenylenyl groups, and heterocyclic aromatic groups disclosed herein. Preferably, “aromatic groups” are benzyl, phenyl, and naphthyl groups, optionally substituted with one or more substitutents.

The term “hydrophobic group,” as used herein, refers to a group having only carbon and hydrogen atoms, optionally substituted with one or more halogen atoms. Preferred hydrophobic groups include, but are not limited to, C₁-C₁₂ straight or branched chain alkyl and haloalkyl groups, C₂-C₁₂ straight or branched chain alkenyl and haloalkenyl groups, C₂-C₁₂ straight or branched chain alkynyl and haloalkynyl groups, phenyl, benzyl, naphthyl, anthracenyl, phenanthracenyl, benzanthracenyl, chrysenyl, and triphenylenyl groups, cyclopropyl, cyclobutyl, cyclopentyl, cyclopentenyl, cyclohexyl, cyclohexenyl, cycloheptanyl, and decahydronaphthalenyl groups, and halophenyl groups. A more preferred hydrophobic group is —CF₃.

The term “hydrophilic group,” as used herein refers to a group that can form hydrogen bonds with water. Examples of such hydrophilic groups include, but are not limited to, hydroxyl, nitro, amino, thiol, alcohol, aldehyde, and carboxyl groups.

The term “polar group,” as used herein, refers to a group that either withdraws electrons from or donates electrons to, respectively, the entire molecule. Examples of such polar groups include, but are not limited to, halogen, hydroxyl, nitro, amino, thiol, alcohol, aldehyde, carboxyl, and O-alkyl groups.

The term “hydrogen bond acceptor group,” as used herein, includes, but is not limited to functional groups such as acetyl, acyl, aldehyde, alkyl chloride, alkyl fluoride, alkyne, amidal, amide, amine, amino acid, anhydride, aromatic rings, azide, azo, azoxy, benzoin, carbamate, carbamic acid, carbamoyl, carbonate, carboxylic acid, carboxylic ester, catenane, cyanamide, cyanate, cyanoamine, cyanohydrin, cyclopropane, diazo, diazonium, disulfide, dithioacetal, enamine, enol, ether, hemiacetal, hemiaminal, hemiketal, hemimercaptal, hydrazide, hydrazine, hydrazone, hydroperoxide, hydroxamic acid, hydroxylamine, imide, imine, imidate, isocyanate, isothiocyanate, ketal, ketene, ketenimine, ketone, nitrile, nitro, nitrone, nitroso, oxazone, oxime, peroxide, phosphate, phosphoester, phosphoryl, phosphonyl, quinone, semicarbazone, sulfamide, sulfate, sulfene, sulfide, sulfinate, sulfonic acid, sulfonic ester, sulfite, sulfonamide, sulfone, sulfonate, sulfonic acid, sulfonic anhydride, sulfonyl, sulfoxide, sulfuryl, thioacetal, thioaldehyde, thioamide, thiocarbamate, thiocyanate, thioether, thioketal, thioketone, thiol acid, thiolactam, thiolactone, thiol ester, thiol, thionocarbonate, thionoester, thionoether, thionolactone, thiosulfate, thiourea, urea, xanthate, ylide, and ynamine groups, as well as heterocyclic groups such as acridinyl, azepinyl, azetidinonyl, azetidinyl, azetyl, aziridinyl, azirinyl, azlactonyl, benzothiophenyl, benzotriazolyl, betainyl, chromanyl, cinnolinyl, dehydropyridinyl, diazepinyl, diazetidinonyl, diazinyl, diaziridinyl, diazirinyl, dioxanyl, dihydrofuranyl, dihydropyranyl, dioxolanyl, dithianyl, dithiolanyl, furanyl, furazanyl, imidazolyl, indazolyl, indolazinyl, indolyl, isoquinolinyl, isothiazolyl, isoxazolyl, morpholinyl, oxadiazolyl, oxazetidinyl, oxazinyl, oxaziridinyl, oxazolyl, oxepinyl, oxetanyl, oxetyl, oxiranyl, phenanthridinyl, phenazinyl, phenothiazinyl, phthalazinyl, piperidinyl, piperazinyl, pteridinyl, purinyl, pyranyl, pyrazinyl, pyrazolyl, pyridazinyl, pyridinyl, pyrimidinyl, pyrrolidinonyl, pyrrolidinyl, pyrrolyl, quinazolinyl, quinolinyl, quinolizidinyl, quinoxalinyl, sulfolenyl, tetrahydrofuranyl, tetrahydropyranyl, tetrazinyl, tetrazolyl, thiadiazolyl, thiazinyl, thiazolyl, thiepanyl, thietanyl, thietyl, thiiranyl, thiophenyl, triazinyl, and triazolyl groups.

5.1 The Prodigiosin Chemotype

Prodigiosin is a tripyrrole-based red pigment originally isolated from the bacterium Serratia marcescens. Prodigiosin has the chemical structure shown in formula I (Boger and Patel, 1988. J. Org. Chem. 53:1405-15).

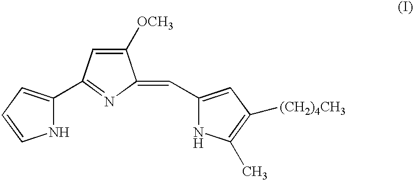

Seriatia mutants and other species of bacteria, including Pseudomonas magnesiorubra, Vibrio psychroerythrus and two Gram-negative, rod-shaped mesophilic marine bacteria that are not members of the genus Serratia, have been found to biosynthesize prodigiosin and other numbers of the prodigiosin family (Gerber (1975) CRC Crit Rev Microbiol. 3(4):469-85). The terms “prodigiosene” and “prodiginine” refer to compounds comprising the common aromatic portion of this molecule (Gerber (1975) CRC Crit Rev Microbiol. 3(4):469-85). Some of these compounds have been shown to possess anti-bacterial activity against several Gram-positive bacteria; some also exhibit anti-malarial activity (Gerber (1975) J. Antibiot. 28: 194-99). At least one member of the prodigiosin family have also been found to act as an immunosuppressant, most likely by decreasing the killing activity of cytotoxic killer T-cells (Nakamura et al. (1989) Transplantation 47(6):1013-6).

As used herein, the phrase “prodigiosin family,” refers to that set of chemical structures that comprise the common three-ring aromatic structure found in prodigiosin as well as those compounds encompassed by the trivial names prodigiosene and prodiginine.

Compounds of the prodigiosin family can be used to treat or prevent cancer or neoplastic disease and to inhibit the growth of neoplastic and cancer cells. The pharmacophores useful in the methods of the present invention, which include anti-apoptotic Bcl protein-inhibitors, were obtained using a prodigiosin chemotype, which encompasses a family of compounds having common structural features and anti-apoptotic Bcl protein inhibition activity.

Compounds of the prodigiosin chemotype utilized of the present invention share the features depicted in formula II.

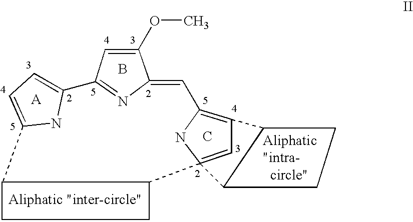

Thus, compounds of the prodigiosin chemotype of formula II, which have Bcl-2 inhibitory activity, possess a core tripyrrole structure (rings A, B and C) and have a methoxyl group at the 3 position of pyrrole ring B. Furthermore, the Bcl-2 inhibiting compounds of this chemotype also have an eleven-carbon, straight or branched chain alkyl group that (a) forms an aliphatic “intra-circle” ring and is bonded at positions 2 and 4 of ring C, (b) forms an aliphatic “inter-circle” ring and is bonded at position 2 on ring C and position 5 on ring A, or (c) does not form a ring, but forms a chain bonded only at position 2 on ring C.

Illustrative compounds of the chemotype of formula II are shown below in Table 1. Undecylprodiginine, butyl-meta-cycloheptylprodiginine (also known as streptorubin B), ethylcyclononyl-prodiginine, ethyl-meta-cyclononyl-prodiginine and methylcyclodecyl-prodiginine have been described in Gerber et al. (1975), Critical Reviews in Microbiology, pp. 469-85.

TABLE 1

| Compound | MW (Daltons) |
|---|---|
| 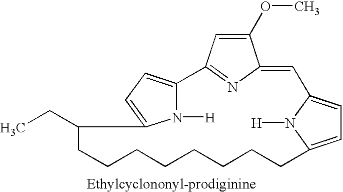 | 391.5 |
| 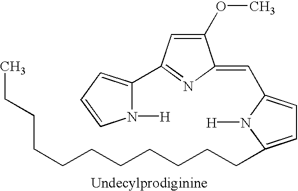 | 393.5 |
| 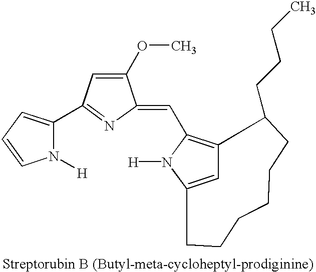 | 391.5 |
| 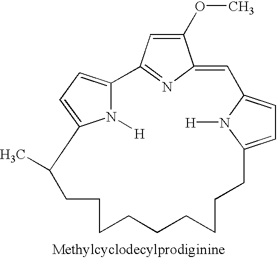 | 391.5 |

5.2 Chemical Structures of Compounds Useful in the Methods of the Present Invention

In one embodiment, compounds useful in the methods of the present invention have a five-membered aromatic heterocycle and a six-membered aromatic ring, as exemplified in Section 6.7 herein. In a preferred embodiment, such compounds have two five-membered aromatic heterocycles, preferably pyrrole rings, that correspond to rings A and B of the general structure of formula II; a hydrophilic or polar substituent at the 3 position of ring B, preferably a methoxyl group; and a hydrophobic, aliphatic or aromatic substituent at position 5 of ring A and at position 2 of ring B.

In another preferred embodiment, compounds useful in the methods of the present invention have three five-membered aromatic heterocycles, preferably pyrrole rings, that correspond to rings A, B and C of the general structure of formula II; a hydrophilic or polar substituent at the 3 position of ring B, preferably a methoxyl group; an aliphatic group at position 5 of ring A; and an aliphatic group at either position 3 or position 4 of ring C.

Computer programs useful for searching databases of chemical compounds useful in the methods of the present invention include ISIS (MDL Information Systems, Inc., San Leandro, Calif.), SYBYL (Tripos, Inc., St. Louis, Mo.), INSIGHT II (Pharmacopeia, Inc., Princeton, N.J.), and MOE (Chemical Computing Group, Inc., Montreal, Quebec, Canada).

Examples of databases of chemical compounds that can be searched using such structure-recognition software include, but are not limited to the BioByte MasterFile (BioByte Corp., Claremont, Calif.), NCI (Laboratory of Medicinal Chemistry, National Cancer Institute, NIH, Frederick, Md.), Derwent (Derwent Information, London, UK) and Maybridge (Maybridge plc, Trevillett, Tintagel, Cornwall, UK) databases, which are available from Pharmacopeia, Inc., Princeton, N.J.).

Specific molecules identified in this manner are further characterized with respect to their ability to inhibit anti-apoptotic:pro-apoptotic protein binding, using, for example, Bcl-2, as an illustrative polypeptide of the anti-apoptotic Bcl protein family and Bax as an illustrative polypeptide of the pro-apoptotic Bcl protein family.

5.2.1 Pharmacophores for Compounds Useful in the Methods of the Present Invention

The present invention is directed toward methods of treating or preventing cancer or neoplastic disease in a patient, comprising administering to a patient in need thereof an effective amount of a compound having the features of a pharmacophore that enable the compound to bind to an anti-apoptotic Bcl protein and prevent homodimer formation and/or to inhibit interactions between an anti-apoptotic Bcl protein and a pro-apoptotic Bcl protein, and thereby kill or inhibit the proliferation of cancer or neoplastic cells, particularly those cancer or neoplastic cells over-expressing an anti-apoptotic Bcl protein. Similarly, the present invention is also directed toward a method for inhibiting the growth of a cancer cell or neoplastic cell, comprising contacting the cancer cell or neoplastic cell with and effective amount of a compound or a pharmaceutically acceptable salt thereof, having the features of such pharmacophores. Accordingly, compounds useful in the methods of the present invention, as described by the pharmacophores disclosed herein, are useful for the treatment and prevention of cancer and neoplastic disease, as well as for inhibiting the growth of cancer cells and neoplastic cells. Compounds having the features of a pharmacophore disclosed herein, where those features have a particular relative orientation represented by the pharmacophore, and that have anti-apoptotic Bcl protein-binding activity, as illustrated by, e.g., in vitro inhibition of proliferation or killing of cancer or neoplastic cells, have therapeutic value. The pharmacophores describe compounds on the basis of chemical features that enable binding interactions between the compound and the chemical substructure(s) within the binding site of the protein (Tomioka et al., (1994) J. Comput. Aided. Mol. Des. 8(4): 347-66; Greene et al. (1994) J. Chem. Inf. Comput. Sci. 34: 1297-1308, which are hereby incorporated by reference in their entireties). Compounds useful in the methods of the present invention therefore, include structurally different compounds that can nevertheless present similar, if not identical, chemical features that are important for interacting with the therapeutic molecule of interest.

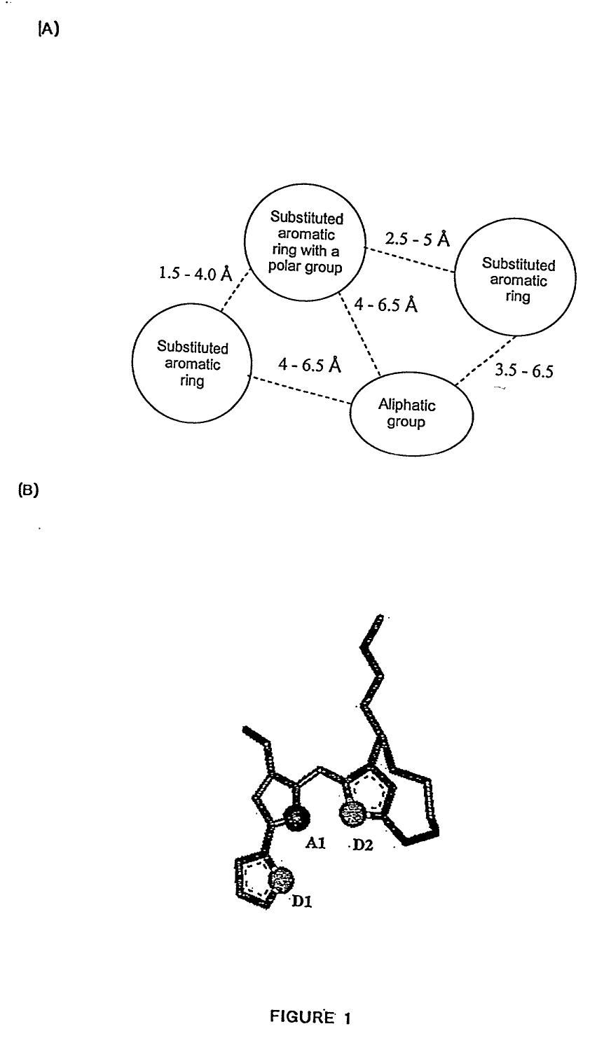

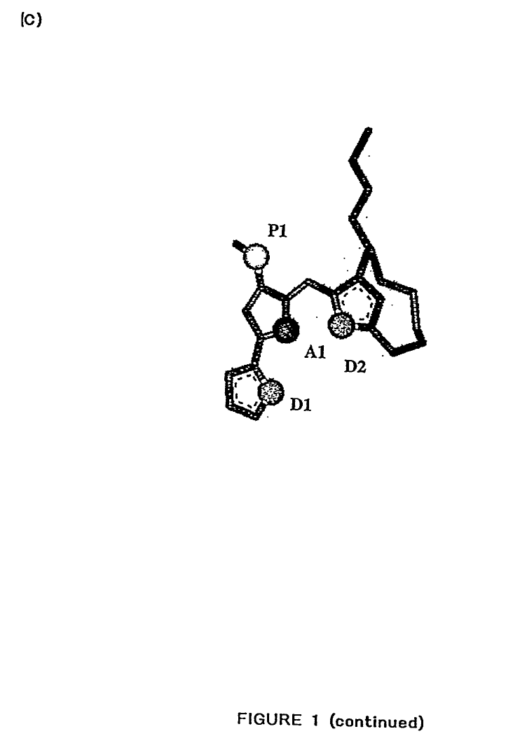

In one embodiment of the present invention, a two-dimensional pharmacophore has the common features of the compounds of the present invention is depicted in FIG. 1A. Ranges of distances between the centroids of each pair of features are listed below in Table 2. The term “centroid” refers to the average spatial position of all of the atoms that are included in that chemical feature. Where n is the number of atoms defining the centroid, and Xᵢ is the position of atom i, the position of the centroid (X_{c}) is calculated as follows:

TABLE 2
${X}_{c} = ( 1 / n ) {\sum }_{i = 1}^{n} {X}_{i}$

| | Range of Distances |
|---|---|
| Features | Between Features (Å) |
| heterocyclic aromatic ring (ring A); | 1.5-4.0 |
| heterocyclic aromatic ring (ring B) | |
| substituted with a polar group | |
| heterocyclic aromatic ring (ring B) | 2.5-5 |
| substituted with a polar group; | |
| heterocyclic aromatic ring (ring C) | |
| heterocyclic aromatic ring (ring B) | 4.0-6.5 |
| substituted with a polar group; | |
| aliphatic group | |
| heterocyclic aromatic ring (ring A); | 4.0-6.5 |
| aliphatic group | |
| heterocyclic aromatic ring (ring C); | 3.5-6.5 |
| aliphatic group | |

It can be predicted that compounds having the chemical features depicted in Table 3, which fall within the scope of the two-dimensional pharmacophore described in Table 2, are anti-apoptotic Bcl protein inhibitors. Accordingly, the structures of Table 3 are used, inter alia, as query structures to search chemical databases for specific molecules that fall within the scope of the two-dimensional pharmacophore. Those specific molecules identified in this manner are then assayed for their ability to inhibit, for example, proliferation of cancer or neoplastic cells, in vivo and/or in vitro, as well as killing of cancer or neoplastic cells, in vivo and/or in vitro.

TABLE 3

| Query Structure | Definition of R Groups |
|---|---|
| 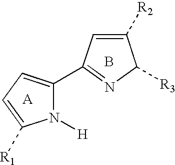 | R₁ is an aliphatic group; R₂ is a hydrophilic or polar group; and R₃ is a hydrophobic group. |
| 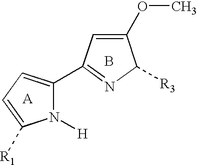 | R₁ is an aliphatic group; and R₃ is a hydrophobic group. |
| 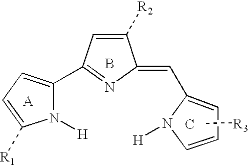 | R₁ and R₃ are each independently an aliphatic group; and R₂ is a hydrophilic or polar group. |
| 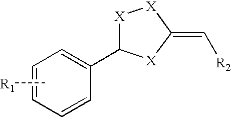 | R₁ and R₂ are each independently an aliphatic, aromatic, hydrophilic or polar group; and each X is independently a carbon, oxygen, sulfur, or nitrogen atom. |
| 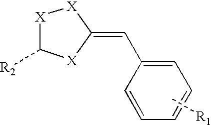 | R₁ and R₂ are each independently an aliphatic, aromatic, hydrophilic or polar group; and each X is independently a carbon, oxygen, sulfur, or nitrogen atom. |
| 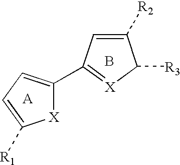 | R₁ is an aliphatic group; R₂ is a hydrophilic or polar group; R₃ is a hydrophobic group or substituted or unsubstituted aromatic group; X_{A} is independently a carbon, oxygen, sulfur, or nitrogen atom; and X_{B} is independently a carbon or nitrogen atom, where X_{A} and X_{B} correspond to X in rings A and B, respectively. |
| 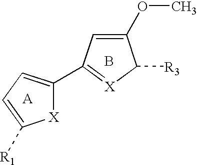 | R₁ is an aliphatic group; R₃ is a hydrophobic group or substituted or unsubstituted aromatic group; X_{A} is independently a carbon, oxygen, sulfur, or nitrogen atom; and X_{B} is independently a carbon or nitrogen atom, where X_{A} and X_{B} correspond to X in rings A and B, respectively. |
| 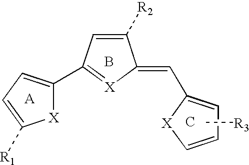 | R₁ and R₃ are each independently an aliphatic group; R₂ is a hydrophilic or polar group; X_{A} and X_{C} are each independently a carbon, oxygen, sulfur, or nitrogen atom; and X_{B} is independently a carbon or nitrogen atom, where X_{A}, X_{B}, and X_{C}, correspond to X in rings A, B, and C, respectively. |

In one embodiment, query structures encompassed by the two-dimensional pharmacophore model of compounds useful in the methods of the present invention have two five-membered aromatic heterocycles, preferably pyrrole rings, that correspond to rings A and B of the general structure of formula II; a hydrophilic or polar substituent at the 3 position of ring B, preferably a methoxyl group, and a hydrophobic, aliphatic or aromatic substituent at position 5 of ring A and at position 2 of ring B.

In another embodiment, query structures, which are encompassed by the two-dimensional pharmacophore model pharmacophore of a compound useful in the methods of the present invention, have three five-membered aromatic heterocycles, preferably pyrrole rings, that correspond to rings A, B and C of the general structure of formula II; a hydrophilic or polar substituent at the 3 position of ring B, preferably a methoxyl group; an aliphatic group at position 5 of ring A; and an aliphatic group at either position 3 or position 4 of ring C.

Therefore, query structures of Table 2 are used to describe features of generic, hypothetical compounds that are used as probes in computer-implemented methods to search chemical databases for compounds useful in the methods of the present invention, which fall within the scope of, for example, a two-dimensional pharmacophore. Computer programs useful for database searching include ISIS (MDL Information Systems, Inc., San Leandro, Calif.), SYBYL (Tripos, Inc., St. Louis, Mo.), INSIGHT II (Pharmacopeia, Princeton, N.J.), and MOE (Chemical Computing Group, Inc., Quebec, Canada).

In another embodiment of the present invention, a three-dimensional pharmacophore of a compound useful in the methods of the present invention has three essential features, as shown in FIG. 1B. As depicted, the pharmacophore consists of a set of features arranged in three-dimensional space. Each feature defines a chemical property of functional groups on molecules.

A hydrogen bond acceptor (“A”) is defined as any atom, including but not limited to, nitrogen, oxygen, and sulfur, having least one available (e.g., nondelocalized) lone electron pair. A hydrogen bond donor (“D”) has available an electropositive hydrogen atom. A polar group (“P”) is defined as a group having a nonzero dipole moment. Complete definitions of these features have been described elsewhere and will easily be understood by those skilled in the art (Greene et al., 1994, J. Chem. Inf. and Comp. Sci. 34:1297-1308).

A three-feature, three-dimensional pharmacophore of a compound useful in the methods of the present invention shown in FIG. 1B, has one hydrogen bond acceptor (“A1”) and two hydrogen bond donors (“D1” and “D2”). The centroids of each pair of features are separated by the ranges of distances shown below in Table 4, which define the relative relationship between the features.

TABLE 4

| Pair of features | Distance between the features |
|---|---|
| A1-D1 | 2.5-4.5 Å |
| A1-D2 | 2.5-4.5 Å |
| D1-D2 | 3.5-5.5 Å |

In another embodiment, a three-dimensional pharmacophore of a human Bcl-2 inhibitor also comprises three features: one hydrogen bond donors (“D1”), one hydrogen bond acceptor (“A1”) and one polar group (“P1”). The centroids of each pair of features are separated by the range of distances as shown below in Table 5.

TABLE 5

| Pair of features | Distance between the features |
|---|---|
| A1-D1 | 2.5-4.5 Å |
| P1-D1 | 4.5-6.5 Å |
| P1-A1 | 2.5-4.5 Å |

In yet another embodiment, a three-dimensional pharmacophore of a human Bcl-2 inhibitor comprises four features: two hydrogen bond donors (“D1” and “D2”), one hydrogen bond acceptor (“A1”) and one polar group (“P1”). This four-feature pharmacophore is shown in FIG. 1C. The centroids of each pair of features are separated by the range of distances as shown below in Table 6.

TABLE 6

| Pair of features | Distance between the features |
|---|---|
| A1-D1 | 2.5-4.5 Å |
| A1-D2 | 2.5-4.5 Å |
| D1-D2 | 3.5-5.5 Å |
| P1-D1 | 4.5-6.5 Å |
| P1-A1 | 2.5-4.5 Å |
| P1-D2 | 4.5-6.5 Å |

Thus, if a database is screened using, e.g., a three-feature three-dimensional pharmacophore, compounds will be selected that have the chemical features A1, D1 and D2 separated by the range of distances in Table 4, or that have the chemical features A1, D1 and P1 separated by the range of distances in Table 5. One of skill in the art will readily appreciate that screening a database using the four-feature pharmacophore will result in the selection of a subset of the compounds selected using either of the three-feature pharmacophores due to the presence of the additional feature, P1 or D2, and the additional distance constraints. In addition, narrowing the range of possible distances between feature centroids in the pharmacophores in effect increases the constraints of the pharmacophore and allows for selection of fewer compounds.

As will also be appreciated by one of skill in the art, the two-dimensional pharmacophore of FIG. 1A and Table 2 is more specific than the three-dimensional pharmacophores depicted in Tables 4, 5, and 6 and in FIGS. 1B and 1C. Thus, if, for example, a database is searched using the two-dimensional pharmacophore, identified compounds are likely to have greater structural similarity to the prodigiosin chemotype than compounds identified using either of the more general three-dimensional pharmacophores.

One of skill in the art will further recognize that the pharmacophores useful in the methods of the present invention can be described in ways other than by using distances between pairs of features and that the present invention is intended to encompass these alternative descriptions of the pharmacophores. For example, the relative disposition of features in the three-dimensional pharmacophores can be described using Cartesian coordinates for the centroid of each feature, which are displacements along x, y and z axes and vectors describing the orientation of each feature. The three-feature and four-feature pharmacophores of the methods of the present invention described above are intended to encompass any model, after optimal superposition of the pharmacophores, comprising the identified features and having a root mean square of equivalent features of less than about 3 Å. More preferably, the pharmacophores encompass any model comprising the identified features and having a root mean square of equivalent features of less than about 1.5 Å, and most preferably, less than about 1.0 Å.

Use of the pharmacophores described in this section to search a chemical database and compounds identified by these searches are described below in Example 6.6.

5.2.2 Computer-implemented Methods for Identifying Compounds Useful in the Methods of the Present Invention that are Anti-apoptotic Bcl Protein Inhibitors

Compounds useful in the methods of the present invention are identified in certain embodiments using computer-assisted methods that detect potential inhibitors of an anti-apoptotic Bcl protein. Such methods can comprise accessing a database of compounds, the database containing structural information about the compounds in the database and comparing the compounds in the database, or a subset of the compounds in the database, with the pharmacophore described above; selecting compounds having the features of the pharmacophore; and outputting information associated with selected compounds, e.g., three dimensional coordinates for each atom of the selected compounds.

Such structural comparisons can be carried out using the software described above, generally using the default parameters supplied by the manufacturer. Such parameters, however, can be modified where desired. For example, when using the MOE-FlexAlign program, the rmsd tolerance can be decreased to 0.1 Å and the failure limit can be decreased to 10. The rmsd tolerance is defined as follows: two configurations are judged as equal if their optimal heavy atom RMS (root mean square) superposition distance is less than the specified value. The failure limit specifies the number of attempts to be made by the software to generate a new alignment before that search is abandoned. Therefore, as one skilled in the art would appreciate, the number of hits to be found in a given database may be influenced by the nature of the pharmacophore or query structure used, the software employed, and the constraints applied to the searches performed by that software.

The computer-assisted methods used in combination with the pharmacophores described above provide those skilled in the art with a tool for identifying compounds, including anti-apoptotic Bcl protein-nhibitors, that can then be evaluated for activity, either in vivo or in vitro. For example, those skilled in the art can use the pharmacophores disclosed herein in conjunction with a computational computer program, such as CATALYST (Molecular Simulations, Inc., San Diego, Calif.), to search databases of existing compounds for compounds that fit the pharmacophores disclosed herein and that, therefore, have an anti-apoptotic Bcl protein-inhibitory activity. “Fit” is used herein to denote the correspondence between some or all of the chemical substructures of an experimental compound to the features of the pharmacophore. The degree of fit of an experimental compound structure to the pharmacophore is calculated using computer-assisted methods to determine whether the compound possesses the chemical features of the pharmacophore and whether the features can adopt the necessary three-dimensional arrangement to fit the model. The computer then reports to one skilled in the art which features of the pharmacophore are fit by an experimental compound. A compound “fits” the pharmacophore if it has the features of the pharmacophore. In one aspect of the present invention, selected compounds are those that have a good fit to the pharmacophore. Without being bound by any theory, these selected compounds bind tightly to an anti-apoptotic Bcl protein and inhibit homodimerization or interactions with a pro-apoptotic Bcl protein and are useful for treating conditions, e.g., cancer or neoplastic disease, that are treated or prevented by inhibiting anti-apoptotic Bcl protein function.

The compound being evaluated, as described above, can be novel or known, and, therefore, one of ordinary skill can readily determine if a compound falls within the scope of the present invention. Using the computer-assisted method and the teachings herein, those skilled in the art can predict that a compound that fits to the pharmacophore described above will inhibit an anti-apoptotic Bcl protein. In an alternative embodiment, one skilled in the art can evaluate the ability of a compound to inhibit an anti-apoptotic Bcl protein using the computer-assisted methods of the invention to predict an IC₅₀ value for the compound in, for example, a Bcl-2/Bax binding assay by evaluating the structural similarity between the compound of interest and a database of known structures for which a IC₅₀ values in a specific assay have been experimentally determined.

After identifying a compound as a potential anti-apoptotic Bcl protein-inhibitor from a database using a two-dimensional pharmacophore, the in vitro and/or in vivo anti-apoptotic Bcl protein inhibitory activity of that compound is determined, using, inter alia, the assays described below. In addition, the three-dimensional structure of that compound is identified, e.g., by using three-dimensional x, y, and z coordinates to define the compound from a structural database. Alternatively, the three-dimensional structures of small molecules can be readily determined using methods known to those skilled in the art, including but not limited to, X-ray crystallography, nuclear magnetic resonance, and crystallographic electron microscopy. The structures obtained from structural databases are usually the structures of non-complexed compounds. If the three dimensional structure is not known, one can use one or more computer programs, including but not limited to, CATALYST (Molecular Simulations, Inc., San Diego, Calif.), to predict the three-dimensional structure of the compound. Three-dimensional conformers are generated from a starting structure using software well known in the art such as, but not limited to, the Best or Fast Conformational Analyses (Molecular Simulations, Inc., San Diego, Calif.) in conjunction with a conformational energy set to a range of 0-50 kcal/mol, preferably to 0-35 kcal/mol, and most preferably to 0-20 kcal/mol and the maximum number of conformations set to 100, preferably 175, and most preferably 255. The pharmacophore is then fit to the compound using tools such as, e.g., Compare within the ViewHypothesis workbench (Molecular Simulations, Inc., San Diego, Calif.), to compare the two structures.

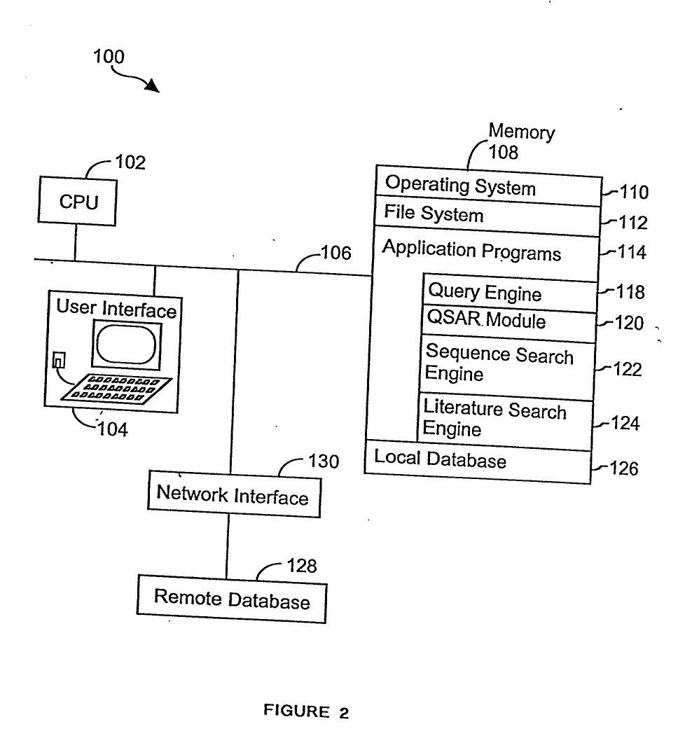

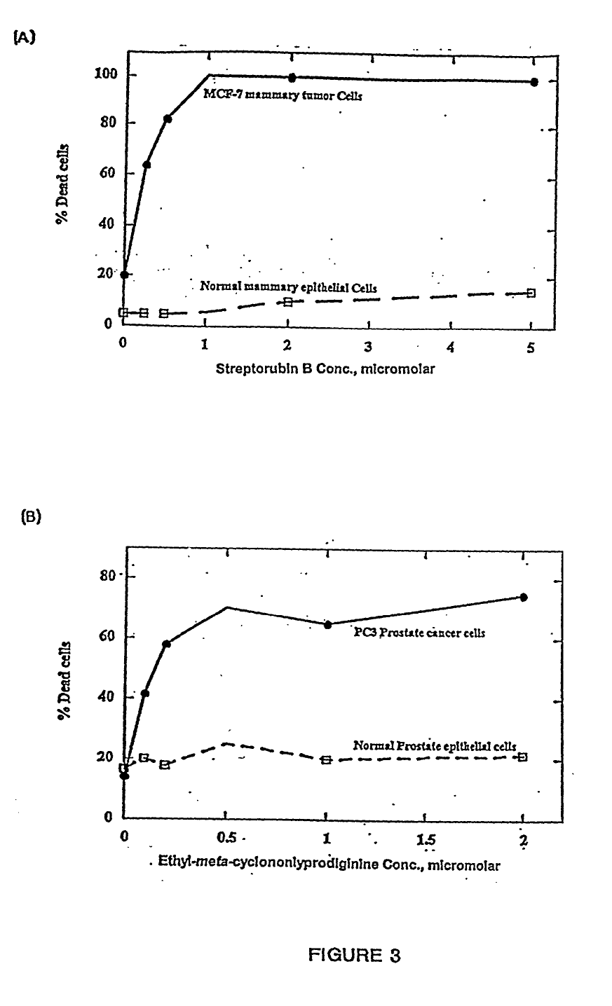

Software-assisted searches of chemical databases for compounds of the present invention can be performed using a wide variety of computer workstations or general purpose computer systems. Referring to FIG. 2, there is shown a computer system 100 on which the method of the present invention can be carried out. A central processing unit 102 is connected via at least one bus 106 to a user interface 104, including one or more input devices such as a keyboard and/or pointer device, and one or more output devices such as a CRT or LCD type display device, and a memory 108. Memory 108 can comprise read-only, or random-access memory, or can comprise “persistent memory” such as may be used for long-term data storage. Stored in memory 108 are an operating system 110, a file system 112, application programs 114 and at least one local database 126. The local database can comprise chemical structure data and/or chemical formula data. Application programs 114 can include but are not limited to a query engine 118, a QSAR module in which is imbedded a pharmacophore 120, a structure search engine 122 and a literature search engine 124. System 100 also comprises a connection via a network interface 130 to at least one remote database 128.

5.3 In Vitro and In Vivo Assays for Proliferation Inhibition and/or Killing of Cancer or Neoplastic Cells

The compounds of the present invention can be shown to inhibit tumor cell proliferation, cell transformation and tumorigenesis in vitro and in vivo using a variety of assays known in the art, or described herein. Such assays can use cells of a cancer cell line, or cells from a patient. Many assays well-known in the art can be used to assess such survival and/or growth; for example, cell proliferation can be assayed by measuring (³H)-thymidine incorporation, by direct cell count, by detecting changes in transcription, translation or activity of known genes such as proto-oncogenes (e.g., fos, myc) or cell cycle markers (Rb, cdc2, cyclin A, D1, D2, D3, E, etc.). The levels of such protein and mRNA and activity can be determined by any method well known in the art. For example, protein can be quantitated by known immunodiagnostic methods such as Western blotting or immunoprecipitation using commercially available antibodies (for example, many cell cycle marker antibodies are available from Santa Cruz Biotechnology, Inc., Santa Cruz, Calif. mRNA can be quantitated by methods that are well known and routine in the art, for example, by Northern analysis, RNase protection, and the polymerase chain reaction in connection with the reverse transcription. Cell viability can be assessed by using trypan-blue staining or other cell death or viability markers known in the art. Differentiation can be assessed, for example, visually based on changes in morphology, etc.

Cell cycle and cell proliferation analysis can be performed using a variety of techniques known in the art, including but not limited to the following:

As one example, bromodeoxyuridine (BRDU) incorporation may be used as an assay to identify proliferating cells. The BRDU assay identifies a cell population undergoing DNA synthesis by incorporation of BRDU into newly synthesized DNA. Newly synthesized DNA can then be detected using an anti-BRDU antibody (see Hoshino et al., 1986, Int. J. Cancer 38, 369; Campana et al., 1988, J. Immunol. Meth. 107, 79).

Cell proliferation can also be examined using (³H)-thymidine incorporation (see e.g., Chen, J., 1996, Oncogene 13:1395-403; Jeoung, J., 1995, J. Biol. Chem. 270:18367-73). This assay allows for quantitative characterization of S-phase DNA synthesis. In this assay, cells synthesizing DNA incorporate (³H)-thymidine into newly synthesized DNA. Incorporation can then be measured using standard techniques in the art such as by counting of radioisotope in a Scintillation counter (e.g. Beckman LS 3800 Liquid Scintillation Counter).

Detection of proliferating cell nuclear antigen (PCNA) can also be used to measure cell proliferation. PCNA is a 36 kilodalton protein whose expression is elevated in proliferating cells, particularly in early G1 and S phases of the cell cycle and therefore can serve as a marker for proliferating cells. Positive cells are identified by immunostaining using an anti-PCNA antibody (see Li et al., 1996, Curr. Biol. 6:189-199; Vassilev et al., 1995, J. Cell Sci. 108:1205-15).

Cell proliferation can be measured by counting samples of a cell population over time (e.g. daily cell counts). Cells may be counted using a hemacytometer and light microscopy (e.g. HyLite hemacytometer, Hausser Scientific). Cell number may be plotted against time in order to obtain a growth curve for the population of interest. In a preferred embodiment, cells counted by this method are first mixed with the dye Trypan-blue, such that living cells exclude the dye, and are counted as viable members of the population.

DNA content and/or mitotic index of the cells can be measured, for example, based on the DNA ploidy value of the cell. For example, cells in the GI phase of the cell cycle generally contain a 2N DNA ploidy value. Cells in which DNA has been replicated but have not progressed through mitosis (e.g. cells in S-phase) exhibit a ploidy value higher than 2N and up to 4N DNA content. Ploidy value and cell-cycle kinetics can be further measured using propidum iodide assay (see e.g. Turner, T., et al., 1998, Prostate 34:175-81). Alternatively, the DNA ploidy can be determined by quantitation of DNA Feulgen staining (which binds to DNA in a stoichiometric manner) on a computerized microdensitometrystaining system (see e.g., Bacus, S., 1989, Am. J. Pathol.135:783-92). In an another embodiment, DNA content can be analyzed by preparation of a chromosomal spread (Zabalou, S., 1994, Hereditas.120:127-40; Pardue, 1994, Meth. Cell Biol. 44:333-351).

The expression of cell-cycle proteins (e.g., CycA, CycB, CycE, CycD, cdc2, Cdk4/6, Rb, p21, p27, etc.) provide crucial information relating to the proliferative state of a cell or population of cells. For example, identification in an anti-proliferation signaling pathway can be indicated by the induction of p21^{cipl}. Increased levels of p21 expression in cells results in delayed entry into G1 of the cell cycle (Harper et al., 1993, Cell 75:805-816; Li et al., 1996, Curr. Biol. 6:189-199). p21 induction can be identified by immunostaining using a specific anti-p21 antibody available commercially (e.g. Santa Cruz Biotechnology, Inc., Santa Cruz, Calif.). Similarly, cell-cycle proteins may be examined by Western blot analysis using commercially available antibodies. In another embodiment, cell populations are synchronized prior to detection of a cell cycle protein. Cell cycle proteins can also be detected by FACS (fluorescence-activated cell sorter) analysis using antibodies against the protein of interest.

Detection of changes in length of the cell cycle or speed of cell cycle can also be used to measure inhibition of cell proliferation by the compounds identified using the pharmacophore of the present invention. In one embodiment the length of the cell cycle is determined by the doubling time of a population of cells (e.g., using cells contacted or not contacted with one or more compounds identified using the pharmacophores of the present invention). In another embodiment, FACS analysis is used to analyze the phase of cell cycle progression, or purify G1, S, and G2/M fractions (see e.g., Delia, D. et al., 1997, Oncogene 14:2137-47).

Lapse of cell cycle checkpoint(s), and/or induction of cell cycle checkpoint(s), can be examined by the methods described herein, or by any method known in the art. Without limitation, a cell cycle checkpoint is a mechanism which ensures that a certain cellular events occur in a particular order. Checkpoint genes are defined by mutations that allow late events to occur without prior completion of an early event (Weinert, T., and Hartwell, L., 1993, Genetics, 134:63-80). Induction or inhibition of cell cycle checkpoint genes can be assayed, for example, by Western blot analysis, or by immunostaining, etc. Lapse of cell cycle checkpoints may be further assessed by the progression of a cell through the checkpoint without prior occurrence of specific events (e.g. progression into mitosis without complete replication of the genomic DNA).

In addition to the effects of expression of a particular cell cycle protein, activity and post-translational modifications of proteins involved in the cell cycle can play an integral role in the regulation and proliferative state of a cell. The invention provides for assays involved detected post-translational modifications (e.g. phosphorylation) by any method known in the art. For example, antibodies that detect phosphorylated tyrosine residues are commercially available, and can be used in Western blot analysis to detect proteins with such modifications. In another example, modifications such as myristylation, can be detected on thin layer chromatography or reverse phase HPLC (see e.g., Glover, C., 1988, Biochem. J. 250:485-91; Paige, L., 1988, Biochem J.; 250:485-91).

Activity of signaling and cell cycle proteins and/or protein complexes is often mediated by a kinase activity. The present invention provides for analysis of kinase activity by assays such as the histone H1 assay (see e.g., Delia, D. et al., 1997, Oncogene 14:2137-47).

The compounds useful in the methods of the present invention can also be demonstrated to alter cell proliferation in cultured cells in vitro using methods which are well known in the art. Specific examples of cell culture models include, but are not limited to, for lung cancer, primary rat lung tumor cells (Swafford et al., 1997, Mol. Cell. Biol., 17:1366-1374) and large-cell undifferentiated cancer cell lines (Mabry et al., 1991, Cancer Cells, 3:53-58); colorectal cell lines for colon cancer (Park and Gazdar, 1996, J. Cell Biochem. Suppl. 24:131-141); multiple established cell lines for breast cancer (Hambly et al., 1997, Breast Cancer Res. Treat. 43:247-258; Gierthy et al., 1997, Chemosphere 34:1495-1505; Prasad and Church, 1997, Biochem. Biophys. Res. Commun. 232:14-19); a number of well-characterized cell models for prostate cancer (Webber et al., 1996, Prostate, Part 1, 29:386-394; Part 2, 30:58-64; and Part 3, 30:136-142; Boulikas, 1997, Anticancer Res. 17:1471-1505); for genitourinary cancers, continuous human bladder cancer cell lines (Ribeiro et al., 1997, Int. J. Radiat. Biol. 72:11-20); organ cultures of transitional cell carcinomas (Booth et al., 1997, Lab Invest. 76:843-857) and rat progression models (Vet et al., 1997, Biochim. Biophys Acta 1360:39-44); and established cell lines for leukemias and lymphomas (Drexler, 1994, Leuk. Res. 18:919-927, Tohyama, 1997, Int. J. Hematol. 65:309-317).

The compounds useful in the methods of the present invention can also be demonstrated to inhibit cell transformation (or progression to malignant phenotype) in vitro. In this embodiment, cells with a transformed cell phenotype are contacted with one or more compounds of the present invention, and examined for change in characteristics associated with a transformed phenotype (a set of in vitro characteristics associated with a tumorigenic ability in vivo), for example, but not limited to, colony formation in soft agar, a more rounded cell morphology, looser substratum attachment, loss of contact inhibition, loss of anchorage dependence, release of proteases such as plasminogen activator, increased sugar transport, decreased serum requirement, or expression of fetal antigens, etc. (see Luria et al., 1978, General Virology, 3d Ed., John Wiley & Sons, New York, pp. 436-446).

Loss of invasiveness or decreased adhesion may also be used to demonstrate the anti-cancer effects of the compounds useful in the methods of the present invention. For example, a critical aspect of the formation of a metastatic cancer is the ability of a precancerous or cancerous cell to detach from primary site of disease and establish a novel colony of growth at a secondary site. The ability of a cell to invade peripheral sites is reflective of a potential for a cancerous state. Loss of invasiveness may be measured by a variety of techniques known in the art including, for example, induction of E-cadherin-mediated cell-cell adhesion. Such E-cadherin-mediated adhesion can result in phenotypic reversion and loss of invasiveness (Hordijk et al., 1997, Science 278:1464-66).

Loss of invasiveness may further be examined by inhibition of cell migration. A variety of 2-dimensional and 3-dimensional cellular matrices are commercially available (Calbiochem-Novabiochem Corp. San Diego, Calif.). Cell migration across or into a matrix may be examined by microscopy, time-lapsed photography or videography, or by any method in the art allowing measurement of cellular migration. In a related embodiment, loss of invasiveness is examined by response to hepatocyte growth factor (HGF). HGF-induced cell scattering is correlated with invasiveness of cells such as Madin-Darby canine kidney (MDCK) cells. This assay identifies a cell population that has lost cell scattering activity in response to HGF (Hordijk et al., 1997, Science 278:1464-66).

Alternatively, loss of invasiveness may be measured by cell migration through a chemotaxis chamber (Neuroprobe/Precision Biochemicals Inc., Vancouver, BC). In such assay, a chemo-attractant agent is incubated on one side of the chamber (e.g., the bottom chamber) and cells are plated on a filter separating the opposite side (e.g., the top chamber). In order for cells to pass from the top chamber to the bottom chamber, the cells must actively migrate through small pores in the filter. Checkerboard analysis of the number of cells that have migrated may then be correlated with invasiveness (see e.g., Ohnishi, T., 1993, Biochem. Biophys. Res. Commun.193:518-25).

The compounds useful in the methods of the present invention can also be demonstrated to inhibit tumor formation in vivo. A vast number of animal models of hyperproliferative disorders, including tumorigenesis and metastatic spread, are known in the art (see Table 317-1, Chapter 317, “Principals of Neoplasia,” in Harrison's Principals of Internal Medicine, 13th Edition, Isselbacher et al., eds., McGraw-Hill, New York, p. 1814, and Lovejoy et al., 1997, J. Pathol. 181:130-135). Specific examples include for lung cancer, transplantation of tumor nodules into rats (Wang et al., 1997, Ann. Thorac. Surg. 64:216-219) or establishment of lung cancer metastases in SCID mice depleted of NK cells (Yono and Sone, 1997, Gan To Kagaku Ryoho 24:489-494); for colon cancer, colon cancer transplantation of human colon cancer cells into nude mice (Gutman and Fidler, 1995, World J. Surg. 19:226-234), the cotton top tamarin model of human ulcerative colitis (Warren, 1996, Aliment. Pharmacol. Ther. 10 Supp 12:45-47) and mouse models with mutations of the adenomatous polyposis tumor suppressor (Polakis, 1997, Biochim. Biophys. Acta 1332:F127-F147); for breast cancer, transgenic models of breast cancer (Dankort and Muller, 1996, Cancer Treat. Res. 83:71-88; Amundadittir et al., 1996, Breast Cancer Res. Treat. 39:119-135) and chemical induction of tumors in rats (Russo and Russo, 1996, Breast Cancer Res. Treat. 39:7-20); for prostate cancer, chemically-induced and transgenic rodent models, and human xenograft models (Royai et al., 1996, Semin. Oncol. 23:35-40); for genitourinary cancers, induced bladder neoplasm in rats and mice (Oyasu, 1995, Food Chem. Toxicol 33:747-755) and xenografts of human transitional cell carcinomas into nude rats (Jarrett et al., 1995, J. Endourol. 9:1-7); and for hematopoietic cancers, transplanted allogeneic marrow in animals (Appelbaum, 1997, Leukemia 11 (Suppl. 4):S15-S17). Further, general animal models applicable to many types of cancer have been described, including, but not restricted to, the p53-deficient mouse model (Donehower, 1996, Semin. Cancer Biol. 7:269-278), the Min mouse (Shoemaker et al., 1997, Biochem. Biophys. Acta, 1332:F25-F48), and immune responses to tumors in rat (Frey, 1997, Methods, 12:173-188).

For example, a compound useful in the methods of the present invention can be administered to a test animal, preferably a test animal predisposed to develop a type of tumor, and the test animal subsequently examined for an decreased incidence of tumor formation in comparison with controls not administered the compound identified using the pharmacophores of the present invention. Alternatively, a compound useful in the methods of the present invention can be administered to test animals having tumors (e.g., animals in which tumors have been induced by introduction of malignant, neoplastic, or transformed cells, or by administration of a carcinogen) and subsequently examining the tumors in the test animals for tumor regression in comparison to controls that were not administered the compound.

5.4 Identification of Compounds that are Useful in the Methods of the Present Invention as Anti-apoptotic Bcl-2 Inhibitors that Inhibit Cancer or Neoplastic Cells In Vitro and/or In Vivo

Pharmacophores of the methods of the present invention, as disclosed supra in Section 5.1, and more particularly in Tables 2, 4, 5, and 6, have been used to screen a number of chemical databases for compounds useful in the methods of the present invention that inhibit cancer or neoplastic cells in vitro and/or in vivo. Analysis of such compounds has revealed a class of compounds, which are particularly useful for the treatment or prevention of neoplastic disease and/or useful for inhibiting growth of cancer cells or neoplastic cells in vitro and in vivo, that are represented by the following Formula III:

A—B—X—C (III)

and pharmaceutically acceptable salts thereof, wherein:

A is selected from the group consisting of

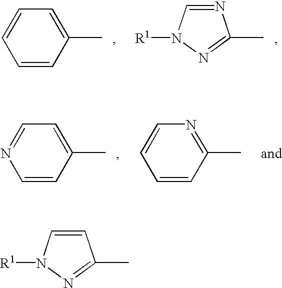

and optionally substituted at one or more carbon atoms with one or more —C₁-C₆, —OC₁-C₆, —OC(O)C₁-C₆, —C(O)C₁-C₆, —C(O)OC₁-C₆, —CF₃, —NO₂, —CH₂O—C₁-C₆, or halo groups;

R¹ is selected from the group consisting of H, —C₁-C₆, and —C(O)C₁-C₆; and optionally substituted at one or more carbon atoms with one or more —C₁-C₆, —OC₁-C₆, —OC(O)C₁-C₆, —C(O)C₁-C₆, —C(O)OC₁-C₆, —CF₃, —NO₂, —CH₂O—C₁-C₆, or halo groups.

X is selected from the group consisting of —O—, —S— and —N(H)—; and

B is selected from the group consisting of

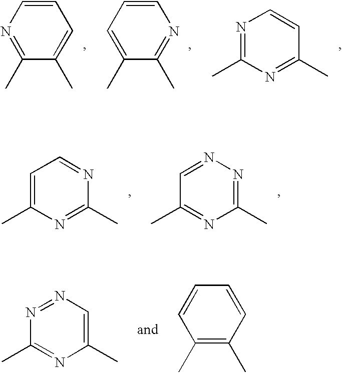

C is selected from the group consisting of

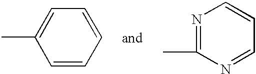

and optionally substituted at one or more carbon atoms with one or more —C₁-C₆, —OC₁-C₆, —OC(O)C₁-C₆, —C(O)C₁-C₆, —C(O)OC₁-C₆, —CF₃, —NO₂, —CH₂O—C₁-C₆, or halo groups.

As shown above, B is selected from a group of radicals forming two bonds: one from the left side of the radical (as shown) and one from the right side. The bond from the left side of each B radical is formed with radical A; the bond from the right side of each B radical is formed with radical X.

5.5 Treatment of Prevention of Cancer or Neoplastic Disease

A compound having the features of a pharmacophore for an anti-apoptotic Bcl protein-inhibitor, or identified using, for example an in vitro or in vivo assay for inhibition or killing of cancer or neoplastic cells, can be used either alone or in combination with other compounds or therapies to treat or prevent cancer or neoplastic disease. In particular, compounds can be used to promote cell death in an anti-apoptotic Bcl protein-overproducing cells.

5.5.1 Therapeutic or Prophylactic Administration of Compounds of the Present Invention

Due to the activity of the compounds, particularly the compounds of Formula III (the “anticancer compounds”), and the pharmaceutically acceptable salts thereof disclosed herein, these compounds are advantageously useful in veterinary and human medicine. For example, the anti-cancer compounds of the present invention are useful for the treatment or prevention of cancer or neoplastic disease or inhibiting the growth of a cancer cell or neoplastic cell.

The anti-cancer compounds are useful for treating or preventing cancer or neoplastic disease in a patient and accordingly, can be used in method for treating or preventing cancer or neoplastic disease in a patient, comprising administering to a patient in need thereof a therapeutically effective amount of an anti-cancer compound. Anti-cancer compounds can be administered for the treatment or prevention of cancer and neoplastic diseases and related disorders including, but not limited to, leukemias such as acute leukemia, acute lymphocytic leukemia, acute myelocytic leukemia, myeloblastic, promyelocytic, myelomonocytic, monocytic, erythroleukemia, chronic leukemia, chronic myelocytic (granulocytic) leukemia, chronic lymphocytic leukemia, and polycythemia vera; lymphomas such as Hodgkin's disease, and non-Hodgkin's disease; multiple myeloma; Waldenström's macroglobulinemia; Heavy chain disease; solid tumors such as sarcomas and carcinomas including fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, stadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

In specific embodiments, cancer, malignancy or dysproliferative changes (such as metaplasias and dysplasias), or hyperproliferative disorders, are treated or prevented in the ovary, breast, colon, lung, skin, pancreas, prostate, bladder, or uterus. In other specific embodiments, sarcoma, melanoma, or leukemia is treated or prevented.

In a preferred embodiment, the anti-cancer compounds are used to treat or prevent cancers including prostate (more preferably hormone-insensitive), neuroblastoma, lymphoma (preferably follicular or diffuse large B-cell), breast (preferably estrogen-receptor positive), colorectal, endometrial, ovarian, lymphoma (preferably non-Hodgkin's), lung (preferably small cell), or testicular (preferably germ cell).

In another preferred embodiment, anti-cancer compounds are used to inhibit the growth of a cell derived from a cancer or neoplasm such as prostate (more preferably hormone-insensitive), neuroblastoma, lymphoma (preferably follicular or diffuse large B-cell), breast (preferably estrogen-receptor positive), colorectal, endometrial, ovarian, lymphoma (preferably non-Hodgkin's), lung (preferably small cell), or testicular (preferably germ cell).

In one embodiment, “treatment” or “treating” refers to an amelioration of a disease, or at least one discernible symptom thereof. In another embodiment, “treatment” or “treating” refers to an amelioration of at least one measurable physical parameter, not necessarily discernible by the patient. In yet another embodiment, “treatment” or “treating” refers to inhibiting the progression of a disease, either physically, e.g., stabilization of a discernible symptom, physiologically, e.g., stabilization of a physical parameter, or both. In yet another embodiment, “treatment” or “treating” refers to delaying the onset of a disease.

In certain embodiments, an anti-cancer compound is administered to a patient, preferably a mammal, more preferably a human, as a preventative measure against cancer or neoplastic disease. As used herein, “prevention” or “preventing” refers to a reduction of the risk of acquiring a disease. In one embodiment, a compound or a pharmaceutically acceptable salt thereof is administered as a preventative measure to a patient.

When administered to a patient, e.g., an animal for veterinary use or to a human for clinical use, or when made to contact a cell or tissue, the anti-cancer compound is preferably in isolated and purified form. By “isolated and purified” it is meant that prior to administration or contacting, a compound is separated from other components of a synthetic organic chemical reaction mixture or natural product source, e.g., plant matter, tissue culture, bacterial broth, etc. Preferably, the anti-cancer compounds are isolated via conventional techniques, e.g., extraction followed by chromatography, recrystallization, or another conventional technique.

The invention provides methods of treatment and prophylaxis by administration to a patient of an effective amount of an anti-cancer. The patient is preferably an animal, including, but not limited to, an animal such a cow, horse, sheep, goat, pig, chicken, turkey, quail, cat, dog, mouse, rat, rabbit, guinea pig, etc., and is more preferably a mammal, and most preferably a human.

The anti-cancer compounds can be administered by any convenient route, for example by infusion or bolus injection, by absorption through epithelial or mucocutaneous linings (e.g., oral mucosa, rectal and intestinal mucosa, etc.) and may be administered together with another biologically active agent. Administration can be systemic or local. Various delivery systems are known, e.g., encapsulation in liposomes, microparticles, microcapsules, and capsules, and can be used to administer an anti-cancer compound. In certain embodiments, more than one anti-cancer compound is administered to a patient. Methods of administration include but are not limited to intradermal, intramuscular, intraperitoneal, intravenous, subcutaneous, intranasal, epidural, oral, sublingual, intranasal, intracerebral, intravaginal, transdermal, rectally, by inhalation, or topically to the ears, nose, eyes, or skin. The preferred mode of administration is left to the discretion of the practitioner, and will depend, in part, upon the site of the medical condition (such as the site of cancer).

In specific embodiments, it may be desirable to administer one or more anti-cancer compounds locally to the area in need of treatment. This may be achieved, for example, and not by way of limitation, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site) of a cancer, tumor or neoplastic or pre-neoplastic tissue.

In certain embodiments, it might be desirable to introduce one or more anti-cancer compounds into the central nervous system by any suitable route, including intraventricular and intrathecal injection. Intraventricular injection may be facilitated by an intraventricular catheter, for example, attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant. In certain embodiments, the anti-cancer compound can be formulated as a suppository, with traditional binders and carriers such as triglycerides.

In another embodiment, the anti-cancer compounds can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In yet another embodiment, the anti-cancer compound can be delivered in a controlled release system. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J. Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled-release system can be placed in proximity of a compound target, e.g., the brain, thus requiring only a fraction of the systemic dose (see, e.g., Goodson, in Medical Applications of Controlled Release, supra, vol.2, pp. 115-138 (1984)). Other controlled-release systems discussed in the review by Langer (Science 249:1527-1533 (1990)) can be used.

Compositions comprising an anti-cancer compound can additionally comprise a suitable amount of a pharmaceutically acceptable vehicle so as to provide the form for proper administration to the patient.

In a specific embodiment, the term “pharmaceutically acceptable” means approved by a regulatory agency of the Federal or a state government or listed in the U.S. Pharmacopoeia or other generally recognized pharmacopoeia for use in animals, and more particularly in humans. The term “carrier” refers to a diluent, adjuvant, excipient, or vehicle with which a compound is administered. Such pharmaceutical carriers can be liquids, such as water and oils, including those of petroleum, animal, vegetable or synthetic origin, such as peanut oil, soybean oil, mineral oil, sesame oil and the like. The pharmaceutical carriers can be saline, gum acacia, gelatin, starch paste, talc, keratin, colloidal silica, urea, and the like. In addition, auxiliary, stabilizing, thickening, lubricating and coloring agents may be used. When administered to a patient, anti-cancer compounds and pharmaceutically acceptable carriers are preferably sterile. Water is a preferred carrier when the anti-cancer compound is administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Such compositions, if desired, can also contain minor amounts of wetting or emulsifying agents, or pH buffering agents.

Such compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable carrier is a capsule (see e.g., U.S. Pat. No. 5,698,155). Other examples of suitable pharmaceutical carriers are described in “Remington's Pharmaceutical Sciences” by E. W. Martin.

In a preferred embodiment, the anti-cancer compounds are formulated in accordance with routine procedures as a pharmaceutical composition adapted for intravenous administration to human beings. Typically, compounds for intravenous administration are solutions in sterile isotonic aqueous buffer. Where necessary, the compositions may also include a solubilizing agent. Compositions for intravenous administration may optionally include a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the anti-cancer compound is to be administered by infusion, it can be dispensed, for example, with an infusion bottle containing sterile pharmaceutical grade water or saline. Where the compound of the present invention is administered by injection, an ampoule of sterile water for injection or saline can be provided so that the ingredients may be mixed prior to administration.

Compositions for oral delivery may be in the form of tablets, lozenges, aqueous or oily suspensions, granules, powders, emulsions, capsules, syrups, or elixirs, for example. Orally administered compositions may contain one or more optionally agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions may be coated to delay disintegration and absorption in the gastrointestinal tract thereby providing a sustained action over an extended period of time. Selectively permeable membranes surrounding an osmotically active driving compound are also suitable for orally administered compounds. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate can also be used. Oral compositions can include standard carriers such as mannitol, lactose, starch, magnesium stearate, sodium saccharine, cellulose, magnesium carbonate, etc. Such carriers are preferably of pharmaceutical grade.

The amount of the anti-cancer compound that will be effective in the treatment of a particular disorder or condition will depend on the nature of the disorder or condition, and can be determined by standard clinical techniques. In addition, in vitro or in vivo assays can optionally be employed to help identify optimal dosage ranges. The precise dose to be employed in the compositions will also depend on the route of administration, and the seriousness of the disease or disorder, and should be decided according to the judgment of the practitioner and each patient's circumstances. However, suitable dosage ranges for intravenous administration are generally about 20-500 micrograms of anti-cancer compound per kilogram body weight. In specific preferred embodiments, the intravenous dose is 10-40, 30-60, 60-100, or 100-200 micrograms per kilogram body weight. In other embodiments, the intravenous dose is 75-150, 150-250, 250-375 or 375-500 micrograms per kilogram body weight. Suitable dosage ranges for intranasal administration are generally about 0.01 pg/kg body weight to 1 mg/kg body weight. Suppositories generally contain active ingredient in the range of 0.5% to 10% by weight. Oral compositions preferably contain 10% to 95% active ingredient. In specific preferred embodiments, suitable dose ranges for oral administration are generally 1-500 micrograms of active compound per kilogram body weight. In specific preferred embodiments, the oral dose is 1-10, 10-30, 30-90, or 90-150 micrograms per kilogram body weight. In other embodiments, the oral dose is 150-250, 250-325, 325-450 or 450-1000 micrograms per kilogram body weight. Effective doses may be extrapolated from dose-response curves derived from in vitro or animal model test systems. Such animal models and systems are well known in the art.

The anti-cancer compounds are preferably assayed in vitro, and then in vivo, for the desired therapeutic or prophylactic activity prior to use in humans. For example, in vitro assays can be used to determine whether administration of a specific compound or combination of compounds is preferred.

In one embodiment, a patient tissue sample is grown in culture and contacted or otherwise administered with an anti-cancer compound, and the effect of such compound upon the tissue sample is observed and compared to a non-contacted tissue. In other embodiments, a cell culture model is used in which the cells of the cell culture are contacted or otherwise administered with an anti-cancer compound, and the effect of such compound upon the tissue sample is observed and compared to a control (non-contacted) cell culture. Generally, a lower level of proliferation or survival of the contacted cells compared to the non-contracted cells indicates that the anti-cancer compound is effective to treat a the patient. Such compounds may also be demonstrated effective and safe using animal model systems.

5.5.2 Treatment or Prevention of Cancer or Neoplastic Disease in Combination with Chemotherapy or Radiotherapy

Cancer or a neoplastic disease, including, but not limited to a neoplasm, a tumor, metastases, or any disease or disorder characterized by uncontrolled cell growth, can be treated or prevented by administration of an anti-cancer compound. Without being bound by any theory, these compounds bind tightly to an anti-apoptotic Bcl protein and inhibit homodimerization or interactions with a pro-apoptotic Bcl protein and are useful for treating conditions, e.g., cancer or neoplastic disease, that are alleviated by inhibition of anti-apoptotic Bcl protein function.

Suitable pharmaceutical compositions can comprise one or more anti-cancer compounds and a pharmaceutically acceptable vehicle.

In certain embodiments an anti-cancer compound is used to treat or prevent cancer or neoplastic disease in combination with one or more anti-cancer, chemotherapeutic agents including, but not limited to, methotrexate, taxol, mercaptopurine, thioguanine, hydroxyurea, cytarabine, cyclophosphamide, ifosfamide, nitrosoureas, cisplatin, carboplatin, mitomycin, dacarbazine, procarbizine, etoposides, campathecins, bleomycin, doxorubicin, idarubicin, daunorubicin, dactinomycin, plicamycin, mitoxantrone, asparaginase, vinblastine, vincristine, vinorelbine, paclitaxel, and docetaxel. In a preferred embodiment, a compound or a pharmaceutically acceptable salt thereof is used to treat or prevent cancer or neoplastic disease in combination with one or more chemotherapeutic or other anti-cancer agents including, but not limited to: γ-radiation; alkylating agents such as cyclophosphamide, ifosfamide, trofosfamide, chlorambucil, carmustine (BCNU), lomustine (CCNU), busulfan, treosulfan, dacarbazine, cisplatin, and carboplatin; plant alkaloids such as vincristine, vinblastine, vindesine, vinorelbine, paclitaxel, and docetaxol; DNA topoisomerase inhibitors such as etoposide, teniposide, topotecan, 9-aminocamptothecin, campto irinotecan, and crisnatol; mytomycins such as mytomycin C; anti-metabolites such as methotrexate, trimetrexate, mycophenolic acid, tiazofurin, ribavirin, EICAR, hydroxyurea, and deferoxamine; pyrimidine analogs such as 5-fluorouracil, floxuridine, doxifluridine, ratitrexed, cytarabine (ara C), cytosine arabinoside, and fludarabine; purine analogs such as mercaptopurine, and thioguanine; hormonal therapies such as tamoxifen, raloxifene, megestrol, leuprolide acetate, flutamide, and bicalutamide; retinoids/deltoids such as vitamin D3 analogs EB 1089, CB 1093 and KH 1060; photodynamic therapies such as vertoporfin (BPD-MA), phthalocyanine, photosensitizer Pc4, and demethoxy-hypocrellin A (2BA-2-DMHA); cytokines such as interferon-α, interferon-γ and tumor necrosis factor; lovastatin; 1-methyl-4-phenylpyridinium ion; staurosporine; actinomycins such as actinomycin D and dactinomycin; bleomycins such as bleomycin A2, bleomycin B2, and peplomycin; anthracyclines such as daunorubicin, doxorubicin (adriamycin), idarubicin, epirubicin, pirarubicin, zorubicin, and mitoxantrone; verapamil; and thapsigargin.

In other embodiments, an anti-cancer compound is administered along with radiation therapy and/or with one or a combination of chemotherapeutic agents, preferably with one or more chemotherapeutic agents with which treatment of the cancer or neoplastic disease has not been found to be refractory. The anti-cancer compound can be administered to a patient that has also undergone surgery as treatment for the cancer.

In another specific embodiment, the invention provides a method for treating or preventing cancer that has shown to be refractory to treatment with a chemotherapy and/or radiation therapy.

In a specific embodiment, an anti-cancer compound is administered concurrently with chemotherapy or radiation therapy. In another specific embodiment, chemotherapy or radiation therapy is administered prior or subsequent to administration of an anti-cancer compound, preferably at least an hour, five hours, 12 hours, a day, a week, a month, more preferably several months (e.g., up to three months), subsequent to administration.

The chemotherapy or radiation therapy administered concurrently with, or prior or subsequent to, the administration of an anti-cancer compound can be accomplished using any method known in the art. The chemotherapeutic agents are preferably administered in a series of sessions, any one or a combination of the chemotherapeutic agents listed above can be administered. With respect to radiation therapy, any radiation therapy protocol can be used depending upon the type of cancer to be treated. For example, but not by way of limitation, x-ray radiation can be administered. In particular, high-energy megavoltage (radiation of greater that 1 MeV energy) can be used for deep tumors, and electron beam and orthovoltage x-ray radiation can be used for skin cancers. Gamma-ray emitting radioisotopes, such as radioactive isotopes of radium, cobalt and other elements, may also be administered to expose tissues to radiation.

Additionally, the invention provides methods for treatment of cancer or neoplastic disease using an anti-cancer compound as an alternative to chemotherapy or radiation therapy where the chemotherapy or the radiation therapy has proven or may prove too toxic, e.g., results in unacceptable or unbearable side effects, for the patient being treated. The patient being treated with the anti-cancer compound can, optionally, be treated with another cancer treatment such as surgery, radiation therapy or chemotherapy, depending on which treatment is found to be acceptable or bearable.

5.5.3 Cancer or Neoplastic Disease Treatable or Preventable According to the Methods of the Present Invention

Cancers or neoplastic diseases and related disorders that can be treated or prevented by administrating an anti-cancer compound include but are not limited to: leukemias such as acute leukemia, acute lymphocytic leukemia, acute myelocytic leukemia, myeloblastic, promyelocytic, myelomonocytic, monocytic, erythroleukemia, chronic leukemia, chronic myelocytic (granulocytic) leukemia, chronic lymphocytic leukemia, and polycythemia vera; lymphomas such as Hodgkin's disease and non-Hodgkin's disease; multiple myeloma; Waldenström's macroglobulinemia; Heavy chain disease; solid tumors such as sarcomas and carcinomas including fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, stadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

In specific embodiments, cancer, malignancy or dysproliferative changes (such as metaplasias and dysplasias), or hyperproliferative disorders, are treated or prevented in the ovary, breast, colon, lung, skin, pancreas, prostate, bladder, or uterus. In other specific embodiments, sarcoma, melanoma, or leukemia is treated or prevented.

In a preferred embodiment, an anti-cancer compound is used to treat or prevent cancers including prostate (more preferably hormone-insensitive), neuroblastoma, lymphoma (preferably follicular or diffuse large B-cell), breast (preferably estrogen-receptor positive), colorectal, endometrial, ovarian, lymphoma (preferably non-Hodgkin's), lung (preferably small cell), or testicular (preferably germ cell).

In another preferred embodiment, an anti-cancer compound is used to inhibit the growth of a cell derived from a cancer or neoplasm such as prostate (more preferably hormone-insensitive), neuroblastoma, lymphoma (preferably follicular or diffuse large B-cell), breast (preferably estrogen-receptor positive), colorectal, endometrial, ovarian, lymphoma (preferably non-Hodgkin's), lung (preferably small cell), or testicular (preferably germ cell).

In specific embodiments of the invention, an anti-cancer compound is used to inhibit the growth of a cell, derived from a cancer or neoplasm, as discussed above in this section.

### 6. EXAMPLES

6.1 Effects of Illustrative Anti-cancer Compounds on Apoptosis of Normal Cells and Cancer Cells

This example describes the effect of illustrative anti-cancer compound compounds of the present invention on apoptosis of normal cells, cancer cells and cells transformed with an oncogene.

### Materials and Methods

Human anti-apoptotic Bcl-2 over-expressing epithelial cells (B23 cells) (Nguyen et al. (1994) J. Biol. Chem. 269 (24): 16521-24) were plated in 24-well plates at a density of about 80,000 cells per well and infected with 12S-adenovirus, which expresses the E1A oncogene (Nguyen et al. (1998) J. Biol. Chem. 273 (50): 33099-102). Normal human breast epithelial cells, MCF-7 breast cancer cells and MBA-MB231 breast cancer cells were plated in 24-well plates at a density of between 70,000-80,000 cells per well. Illustrative anti-cancer compounds were added to the cells at the concentrations indicated in Table 7. After incubation at 37° C. for the indicated times in a 5% CO₂ incubator, cells were harvested and cell death was monitored using a trypan blue assay as described in Ausubel et al. (1988) Current Protocols in Molecular Biology, Section 11.5.1 (Greene Publishing Associates and Intersciences, New York)).

TABLE 7

| | | | Cytotoxicity (% Dead Cells) | | | |
|---|---|---|---|---|---|---|
| | | Time of | | Normal human | | MBA-MB231 |
| | Concentration | Exposure | | breast | MCF-7 breast | breast cancer |
| Compound | (μM) | (h) | B23 cells | epithelial cells | cancer cells | cells |
| Butyl-meta-cyclo- | 0 | 72 | 10 | 5 | 24 | 20 |
| heptylprodiginine | 0.25 | 72 | 75 | 5 | 70 | 64 |
| | 1.0 | 72 | 85 | 5 | 100 | 100 |
| | 2.0 | 72 | 95 | 15 | 100 | 100 |
| Ethylcyclo- | 1.0 | 72 | 15 | n/a | n/a | n/a |
| nonylprodiginine | 5.0 | 72 | 20 | n/a | n/a | n/a |
| Undecyl- | 1.0 | 72 | 18 | n/a | n/a | n/a |
| prodiginine | 5.0 | 72 | 25 | n/a | n/a | n/a |
| Ethyl-meta-cyclo- | 0 | 24 | 8 | 17 | 14 | n/a |
| nonlylprodiginine | 0.2 | 24 | 32 | 10 | 45 | n/a |
| | 1.0 | 24 | 36 | 9 | 62 | n/a |
| | 2.0 | 24 | 48 | 15 | 65 | n/a |

n/a = Data not available

### Results

Normally, intracellular expression of an oncogene in cells results in apoptotic cell death. However, if anti-apoptotic Bcl-2 is over-expressed in these cells, they are protected from apoptosis. Therefore, an anti-cancer compound's activity can be monitored by measuring cell death in B23 cells in the presence of the E1A oncogene. As indicated in Table 7, above, concentrations of butyl-meta-cycloheptylprodiginine as low as 0.25 μM were able to inhibit Bcl-2 in B23 cells and could re-establish the killing effect of the E1A oncogene. In contrast, in the absence of E1A, addition of butyl-meta-cycloheptylprodiginine had no significant effect on B23 cell death (data not shown).

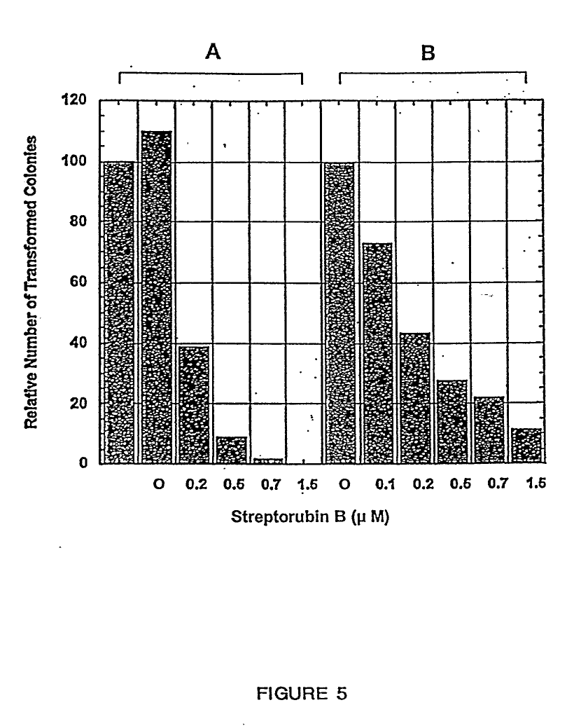

The effect of illustrative anti-cancer compounds on the death of normal or cancerous cells was also monitored. As shown in Table 7, above, and in FIG. 5A, exposure of MCF-7 breast cancer cells to 1 μM butyl-meta-cycloheptylprodiginine resulted in the death of 100% of the cancer cells, whereas normal human breast epithelial cells were not affected. Similarly, exposure of PC3 prostate cancer cells to 0.5-2 μM ethyl-meta-cyclononylprodiginine resulted in significant cell death, whereas exposure of normal prostate epithelial cells to the compound did not induce apoptosis in the normal cells (FIG. 5B).

Accordingly, the results of these cell-killing assays indicate that butyl-meta-cycloheptylprodiginine, ethylcyclo-nonylprodiginine, undecyl-prodiginine and ethyl-meta-cyclo-nonlylprodiginine, illustrative anti-cancer compounds, selectively induce apoptosis in anti-apoptotic Bcl-2 overproducing cancer cell lines without similarly affecting normal tissues. Consequently, anti-cancer compounds are useful for treating or preventing cancer or a neoplastic disease.

6.2 Apoptosis Reinstatement in Anti-apoptotic Bcl-2 Over-expressing Cells Following Contact with Butyl-meta-cycloheptylprodiginine

This example demonstrates the ability of butyl-meta-cycloheptylprodiginine, an illustrative anti-cancer compound, to reinstate apoptosis in a anti-apoptotic Bcl-2 over-expressing cell line transformed with an oncogene.

Without being bound by any theory, anti-cancer compounds are believed to bind tightly to an anti-apoptotic Bcl protein and inhibit homodimerization or interactions with a pro-apoptotic Bcl protein. In this manner, anti-cancer compounds may thereby alleviate inhibition apoptosis by anti-apoptotic Bcl protein(s), consequently inhibiting growth of cancer cells or neoplastic cells in vitro and/or in vivo, in which an anti-apoptotic Bcl protein is over-expressed. Such anti-apoptotic Bcl proteins include, but are not limited to, Bcl-2, Bcl-w, Mcl-1, and Bcl-xl.

### Materials and Methods

Human oral epithelial carcinoma cells (KB cells) with and without stably expressed anti-apoptotic Bcl-2 were infected with 12S-adenovirus expressing the E1A oncogene, as described above in Example 6.1. Butyl-meta-cycloheptylprodiginine was added to the cells immediately after infection at the concentrations indicated in FIG. 6. Cell death was monitored 70 hours after infection using the trypan blue assay described above.

### Results

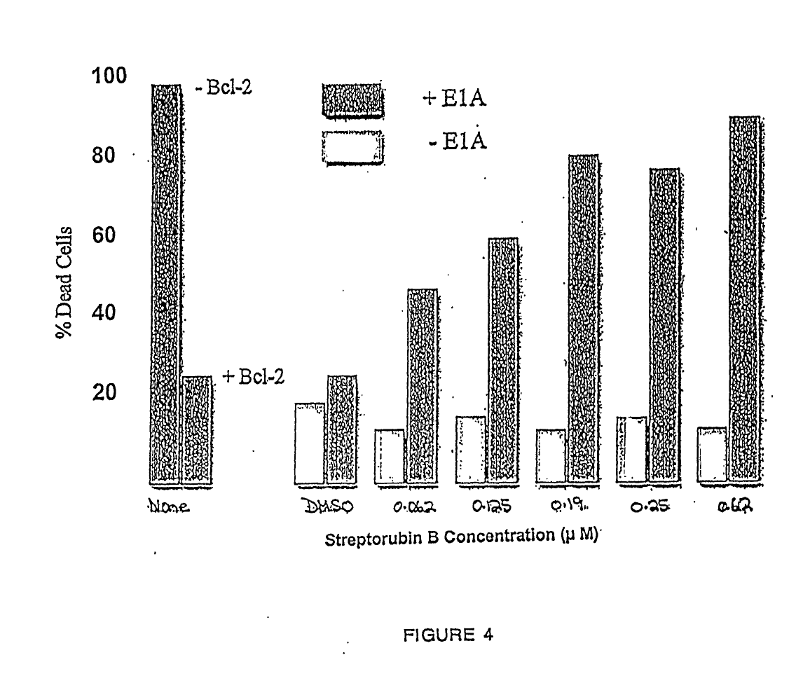

Results of this experiment are shown in FIG. 4. In the absence of Bcl-2, most of the infected KB cells were dead after 70 hours, whereas only about 20% of anti-apoptotic Bcl-2 over-expressing KB cells were dead after the same period of time. Thus, anti-apoptotic Bcl-2 over-expression protects from apoptosis cells that are infected with an oncogene. The addition of various concentrations of butyl-meta-cycloheptylprodiginine reinstate apoptosis in E1A-infected cells (darker gray bars), whereas these concentrations have no effect on apoptosis on uninfected cells (lighter gray bars). Thus, butyl-meta-cycloheptylprodiginine, an illustrative compound of the present invention, induces apoptosis in infected cells and, accordingly is useful for treating or preventing cancer or a neoplastic disease.

6.3 Induction of Apoptosis in Transformed Cells

This example demonstrates the effect of butyl-meta-cycloheptylprodiginine, an illustrative anti-cancer compound, on transformed cells that over produce anti-apoptotic Bcl-2.

### Materials and Methods

Baby rat kidney cells were transfected with RcRSV (Hartl et al. (1992) Cell Growth Differ. 3 (12): 909-18) expressing both E1A oncogene and anti-apoptotic Bcl-2 as described in (Lin et al. (1995) Mol. Cell. Biol. 15 (8): 4536-44). Transfected cells were then cultured (Lin et al. (1995) Mol. Cell. Biol. 15 (8): 4536-44) in the presence of varying concentrations of Butyl-meta-cycloheptylprodiginine for three weeks and the number of transformed cell colonies was counted.

### Results

In the absence of anti-apoptotic Bcl-2, all baby rat kidney cells died within a few days (data not shown). As demonstrated in FIG. 5, 100 colonies of cells that were transfected with anti-apoptotic Bcl-2 and the E1A oncogene were transformed after three weeks (“Con”). The addition of increasing concentrations of butyl-meta-cycloheptylprodiginine to the rat kidney cells significantly lowered the ability of anti-apoptotic Bcl-2 to transform cells in the presence of E1A (FIG. 5A). In addition, the compound promoted apoptosis of the transformed colonies (FIG. 5B). After transformation with anti-apoptotic Bcl-2, the cells became susceptible to butyl-meta-cycloheptylprodiginine, whereas normal baby rat kidney cells were not susceptible to butyl-meta-cycloheptylprodiginine compound under similar conditions (data not shown).

Accordingly, this example demonstrates the ability of butyl-meta-cycloheptylprodiginine, an illustrative anti-cancer compound, to inhibit transformation of cells brought about, in part, by overproduction of anti-apoptotic Bcl-2 and, accordingly, to treat or prevent cancer or a neoplastic disease.

6.4 Cell Proliferation Assays

Cell proliferation was measured using MCF-7 cells, which had been which had been seeded in 96-well plates (approximately 8000 cells/ well) and incubated overnight in RPMI 1640 medium supplemented with 20 μg insulin/ml. The seeded MCF-7 cells were then contacted with compounds dissolved in DMSO or with the solvent alone. Proliferation was monitored at 24 and 48-hour intervals by adding wst-1 dye to stain metabolically active, living cells. After a one hour incubation, unbound dye was removed and the extent of cell staining was determined by measuring light absorption at 450 nm. Results were expressed as the percentage inhibition of cell proliferation after 48 hour incubation, using as a control, cells that had been contacted with the DMSO solvent alone.

6.5 Cell Killing Assays

Cytotoxicity of anti-cancer compounds is determined by contacting cancerous MCF-7 cells, in RPMI 1640 medium (Clonetics Products of BioWhittaker, Inc., Walkersville, Md.) supplemented with 20 μg insulin/ml (Clonetics Products of BioWhittaker, Inc., Walkersville, Md.) and control normal breast epithelial cells in Mammary Epithelial Growth Medium (Clonetics Products of BioWhittaker, Inc., Walkersville, Md.). Cells were plated in 24-well plates (-30,000 cells/well) and contacted either with compounds, dissolved in DMSO, or with solvent alone. Cell killing was monitored at 24, 48 and 72-hour intervals using trypan blue exclusion assay (Ausubel et al. (1988) Current protocols in Molecular Biology, Section 11.5.1 (Greene Publishing Associates and Intersciences, New York)). Results were expressed as the percentage of dead cells within the cell population, after 48 hr incubation.

6.6 Use of Three Feature Three-dimensional Pharmacophore to Search Chemical Databases for Human Anti-apoptotic Bcl-2 Inhibitors

The three-feature pharmacophore features and distances listed above in Table 4 were used to search the Available Chemicals Directory Database using the computer-based methods implemented in ISIS (MDL Information Systems, Inc., San Leandro, Calif.). For example, an initial two-dimensional search was performed using ISIS_Base (MDL Information Systems, Inc., San Leandro, Calif.) in the ACD database using the following query structure, where Q represents any atom except carbon or hydrogen at that position:

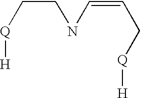

Shown here is a two-dimensional representation (for clarity) of the three-dimensional, three-point pharmacophore query structure used to identify the compounds shown below in this section.

The three-dimensional structures of representative compounds detected were generated using the default parameters of the MOE energy minimizer (Chemical Computing Group, Inc., Montreal, Quebec, Canada), and the exemplary compounds of Table 4, were selected.

The compounds listed below were identified by this method.

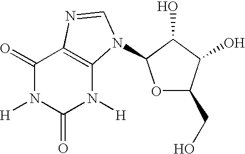

9-(3,4-Dihydroxy-5-hydroxymethyl-tetrahydro-furan-2-yl)-3,9-dihydro-purine-2,6-dione

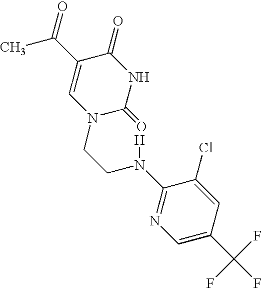

5-Acetyl-1-[2-(3-chloro-5-trifluoromethyl-pyridin-2-ylamino)-ethyl]-1H-pyrimidine-2,4-dione

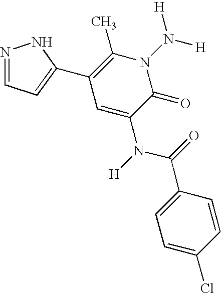

N-[1-Amino-6-methyl-2-oxo-5-(2H-pyrazol-3-yl)-1,2-dihydro-pyridin-3-yl]-4-chloro-benzamide

Accordingly, the pharmacophores disclosed herein can be used as query structures to identify anti-cancer compound compounds, which include anti-apoptotic-Bcl inhibitors, from within the population of molecules of a chemical database. Such anti-apoptotic-Bcl inhibitors induce apoptosis in transformed cells and are useful in the treatment and prevention of cancer and neoplastic diseases.

6.7 Use of a Two-dimensional Pharmacophore to Search a Chemical Database for Human Anti-apoptotic Bcl-2 Inhibitors

The query structures of Table 3, which are representative of the two-dimensional pharmacophore having the features and distances listed above in Table 2, were used to search the ACD chemical database (Available Chemical Directory; MDL Information Systems, Inc., San Leandro, Calif.), including published inhibitors of Bcl-2 and Bcl-X_{L} (Wang et al. 2000, Proc. Natl. Acad. Sci. 97: 7124-29; Degterev et al. 2001, Nature Cell Biol. 3: 173-82), which were added to the database. The structures of the published inhibitors of Bcl-2 and Bcl-X_{L}, as well as the following query structure of Table 3, were drawn using ISIS/DRAW software (MDL Information Systems, San Leandro, Calif.):

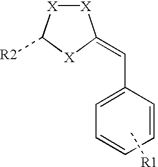

The chemical database was scanned using this drawn query structure using the substructure search function of ISIS/BASE software (MDL Information Systems, Inc., San Leandro, Calif.), and the following compound was detected, which included as part of its structure the query structure representing the disclosed two-dimensional pharmacophore:

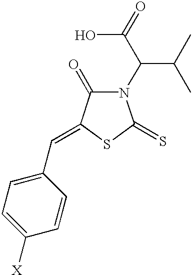

X is H, Br, Cl, N(CH₃)₂

Accordingly, the pharmacophores disclosed herein can be used as query structures to identify anti-cancer compound compounds, which include anti-apoptotic-Bcl inhibitors, from within the population of molecules of a chemical database. Such anti-apoptotic-Bcl inhibitors induce apoptosis in transformed cells and are useful in the treatment and prevention of cancer and neoplastic diseases.

6.8 Identification of Compounds Falling within the Scope of a Pharmacophore

Specific compounds or classes of compounds were analyzed to determine their fit to the pharmacophores disclosed herein. The test molecules, which include published inhibitors of Bcl-2 and Bcl-X_{L} (Wang et al. 2000, Proc. Natl. Acad. Sci. 97: 7124-29; Degterev et al. 2001, Nature Cell Biol. 3: 173-82), were drawn in MOE (Chemical Computing Group, Inc., Quebec, Canada), with a conformational search calculation carried out for each compound in MOE, using the default parameters provided by the software vendor using the SYSTEMATIC SEARCH module. Equivalent conformational search functions are also available in other, commercially available modeling software packages, such as SYBYL (Tripos, Inc., St. Louis, Mo.) or INSIGHT II (Pharmacopoeia, Inc., Princeton, N.J.). For each compound a potential hydrogen bond donor (D1) or donors (D1 and D2), hydrogen bond acceptor (A1), and polar group (P1), were identified using the complete conformational ensemble of each molecule, and the inter-feature distances calculated and compared for their fit to the three-feature, three-dimensional, and four-feature, three-dimensional pharmacophores disclosed herein, in Tables 4, 5, and 6.

The following compounds were identified as falling within the scope of the three-feature, three-dimensional pharmacophore, having the features disclosed in Table 5:

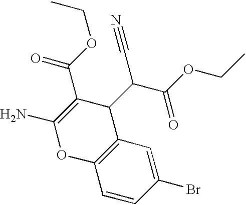

2-amino-6-bromo-4-(cyano-ethoxycarbonyl-methyl)-4H-chromene-3-carboxylic acid ethyl ester

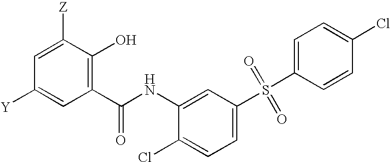

Where Y is Cl and Z is Br; Y is Cl and Z is I; or Y and Z are I.

Accordingly, by using commercially available software, one of ordinary skill in the art would be able to determine if a compound would fall within the scope of a pharmacophore disclosed herein, and, consequently, could identify that compound as potentially useful in the methods disclosed herein.

6.9 Inhibition of Proliferation and Cell-killing Activities of Illustrative Anti-cancer Compounds Useful in the Methods of the Present Invention

This example demonstrates the ability of undecylprodiginine, butyl-meta-cycloheptylprodiginine (also known as streptorubin B), ethylcyclononyl-prodiginine, ethyl-meta-cyclononyl-prodiginine and methylcyclodecyl-prodiginine, which are illustrative anti-cancer compounds, to kill E1A cells, in vitro. IC₅₀ values determined for these illustrative compounds in an E1A killing assay, using the materials and methods described above. These compounds have been described in Gerber et al. (1975), Critical Reviews in Microbiology, pp. 469-85.

TABLE 8

| | | IC₅₀ (μM) or | | |
|---|---|---|---|---|
| | | [% inhibition at μM | | |
| | | concentration] | | IC₅₀ (μM) or |
| | | Anti-apoptotic:Pro-apoptotic | | [% inhibition at μM |
| | MW | binding assay | | concentration] |
| Compound | (Daltons) | Bcl-2 | Bcl-w | E1A-Bcl2 killing assay |
| 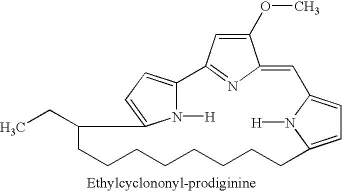 | 391.5 | 45%; 510 μM | n/a | 20%; 5 μM |
| 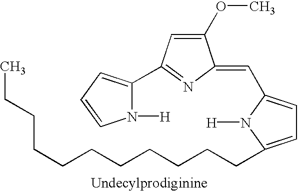 | 393.5 | 40%; 500 μM | n/a | 25%; 5 μM |
| 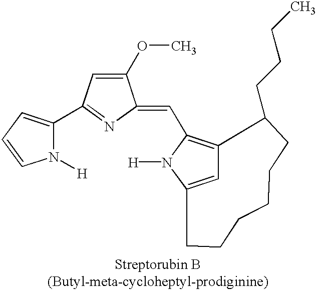 | 391.5 | 125 | 100 | 0.05 |
| 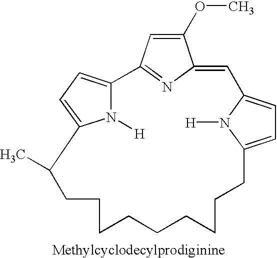 | 391.5 | 510 | 510 | >2 μM |

Accordingly, the data of Table 8 demonstrate that, undecylprodiginine, butyl-meta-cycloheptylprodiginine, ethylcyclononyl-prodiginine, ethyl-meta-cyclononyl-prodiginine and methylcyclodecyl-prodiginine, are capable of killing E1A cells, in vitro. Consequently, the anti-cancer compounds are useful for inhibition of the growth of cancer cells and neoplastic cells and for the prevention and treatment of cancer and neoplastic disease in a patient.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

Various publications are cited herein, the disclosures of which are incorporated by reference in their entireties.

What is claimed is:

1. A method for treating or preventing cancer or neoplastic disease in a patient, comprising administering to a patient in need thereof an effective amount of a compound or a pharmaceutically acceptable salt thereof having the following features:
(a) a first hydrogen bond donor feature, D1;
(b) a hydrogen bond acceptor feature, A1; and
(c) a second hydrogen bond donor feature, D2;
wherein said D1, A1 and D2 each has a centroid, each centroid being separated by the distances:

| Pair of features | Distance between the features |
|---|---|
| A1-D1 | 2.5-4.5 Å |
| A1-D2 | 2.5-4.5 Å |
| D1-D2 | 3.5-5.5 Å. |

2. The method of claim 1, wherein said cancer or neoplastic disease is selected from the group consisting of acute leukemia, acute lymphocytic leukemia, acute myelocytic leukemia, myeloblastic, promyelocytic, myelomonocytic, monocytic, erythroleukemia, chronic leukemia, chronic myelocytic (granulocytic) leukemia, chronic lymphocytic leukemia, polycythemia Vera, Hodgkin's disease, non-Hodgkin's disease; multiple myeloma, Waldenström's macroglobulinemia, heavy chain disease, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, stadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, and endometrial cancer.

3. The method of claim 1, wherein the cancer or neoplastic disease over-expresses an anti-apoptotic Bcl protein.

4. The method of claim 3, wherein the anti-apoptotic Bcl protein is selected from the group consisting of Bcl-2, Bcl-w, Mcl-1, and Bcl-xl.

5. A method for treating or preventing cancer or neoplastic disease in a patient, comprising administering to a patient in need thereof an effective amount of a compound or a pharmaceutically acceptable salt thereof having the following features:
(a) a hydrogen bond donor feature, D1;
(b) a hydrogen bond acceptor feature, A1; and
(c) a polar group feature, P1;
wherein said D1, A1 and P1 each has a centroid, each centroid being separated by the distances:

| Pair of features | Distance between the features |
|---|---|
| A1-D1 | 2.5-4.5 Å |
| P1-D1 | 4.5-6.5 Å |
| P1-A1 | 2.5-4.5 Å. |

6. The method of claim 5, wherein said cancer or neoplastic disease is selected from the group consisting of acute leukemia, acute lymphocytic leukemia, acute myelocytic leukemia, myeloblastic, promyelocytic, myelomonocytic, monocytic, erythroleukemia, chronic leukemia, chronic myelocytic (granulocytic) leukemia, chronic lymphocytic leukemia, polycythemia vera, Hodgkin's disease, non-Hodgkin's disease; multiple myeloma, Waldenström's macro globulinemia, heavy chain disease, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, stadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, and endometrial cancer.

7. The method of claim 5, wherein the cancer or neoplastic disease over-expresses an anti-apoptotic Bcl protein.

8. The method of claim 7, wherein the anti-apoptotic Bcl protein is selected from the group consisting of Bcl-2, Bcl-w, Mcl-1, and Bcl-xl.

9. A method for treating or preventing cancer or neoplastic disease in a patient, comprising administering to a patient in need thereof an effective amount of a compound or a pharmaceutically acceptable salt thereof having the following features:
(a) a heterocyclic aromatic ring, Ring A;
(b) a heterocyclic aromatic ring, Ring B, substituted with a polar group;
(c) a heterocyclic aromatic ring, Ring C;
(d) an aliphatic group:
wherein said heterocyclic aromatic ring, Ring A; heterocyclic aromatic ring, Ring B, substituted with a polar group; heterocyclic aromatic ring, Ring C; and aliphatic group, each have a centroid, each centroid being separated by the distances:

| | Range of Distances |
|---|---|
| Features | Between Features (Å) |
| heterocyclic aromatic ring (ring A); | 1.5-4.0 |
| heterocyclic aromatic ring (ring B) | |
| substituted with a polar group | |
| heterocyclic aromatic ring (ring B) | 2.5-5 |
| substituted with a polar group; | |
| heterocyclic aromatic ring (ring C) | |
| heterocyclic aromatic ring (ring B) | 4.0-6.5 |
| substituted with a polar group; | |
| aliphatic group | |
| heterocyclic aromatic ring (ring A); | 4.0-6.5 |
| aliphatic group | |
| heterocyclic aromatic ring (ring C); | 3.5-6.5 |
| aliphatic group | |

10. The method of claim 9, wherein said cancer or neoplastic disease is selected from the group consisting of acute leukemia, acute lymphocytic leukemia, acute myelocytic leukemia, myeloblastic, promyelocytic, myelomonocytic, monocytic, erythroleukemia, chronic leukemia, chronic myelocytic (granulocytic) leukemia, chronic lymphocytic leukemia, polycythemia vera, Hodgkin's disease, non-Hodgkin's disease; multiple myeloma, Waldenström's macroglobulinemia, heavy chain disease, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, stadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, and endometrial cancer.

11. The method of claim 9, wherein the cancer or neoplastic disease over-expresses an anti-apoptotic Bcl protein.

12. The method of claim 11, wherein the anti-apoptotic Bcl protein is selected from the group consisting of Bcl-2, Bcl-w, Mcl-1, and Bcl-xl.

13. A method of treating or preventing cancer or neoplastic disease, comprising administering to a patient in need thereof an effective amount of a compound or a pharmaceutically acceptable salt thereof having the following features:
(a) a first hydrogen bond donor feature, D1;
(b) a hydrogen bond acceptor feature, A1;
(c) a second hydrogen bond donor feature, D2; and
(d) a polar group feature, P1;
wherein said D1, A1, D2 and P1 each has a centroid, each centroid being separated by the distances:

| Pair of features | Distance between the features |
|---|---|
| A1-D1 | 2.5-4.5 Å |
| A1-D2 | 2.5-4.5 Å |
| D1-D2 | 3.5-5.5 Å |
| P1-D1 | 4.5-6.5 Å |
| P1-A1 | 2.5-4.5 Å |
| P1-D2 | 4.5-6.5 Å. |

14. The method of claim 13, wherein said cancer or neoplastic disease is selected from the group consisting of acute leukemia, acute lymnphocytic leukemia, acute myelocytic leukemia, myeloblastic, promyelocytic, myelomonocytic, monocytic, erytliroleukemia, chronic leukemia, chronic myelocytic (granulocytic) leukemia, chronic lymphocytic leukemia, polycythemia vera, Hodgkin's disease, non-Hodgkin's disease; multiple myeloma, Waldenström's macroglobulinemia, heavy chain disease, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, stadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, and endometrial cancer.

15. The method of claim 13, wherein the cancer cell or neoplastic cell over-expresses an anti-apoptotic Bcl protein.

16. The method of claim 15, wherein the anti-apoptotic Bcl protein is selected from the group consisting of Bcl-2, Bcl-w, Mcl-1, and Bcl-xl.

17. A method for treating or preventing cancer or neoplastic disease in a patient, comprising administering to a patient in need thereof an effective amount of a compound of Formula III:
A—B—X—C (III)
or a pharmaceutically acceptable salt thereof,
wherein:
A is selected from the group consisting of 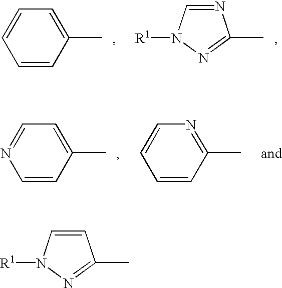
and optionally substituted at one or more carbon atoms with one or more —C₁-C₆, —OC₁-C₆, —OC(O)C₁-C₆, —C(O)C₁-C₆, —C(O)OC₁-C₆, —CF₃, —NO₂, —CH₂O—C₁-C₆, or halo groups;
R¹ is selected from the group consisting of H, —C₁-C₆ and —C(O)C₁-C₆;
B is selected from the group consisting of 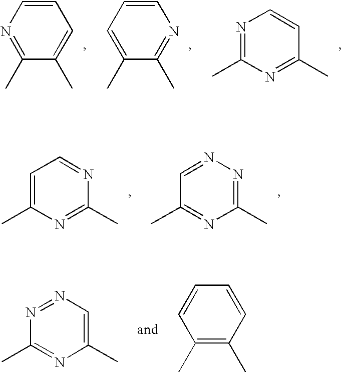
and optionally substituted at one or more carbon atoms with one or more —C₁-C₆, —OC₁-C₆, —OC(O)C₁-C₆, —C(O)C₁-C₆, —C(O)OC₁-C₆, —CF₃, —NO₂, —CH₂O—C₁-C₆, or halo groups.
X is selected from the group consisting of —O—, —S— and —N(H)—; and
C is selected from the group consisting of 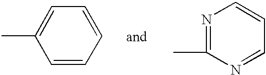
and optionally substituted at one or more carbon atoms with one or more —C₁-C₆, —OC₁-C₆, —OC(O)C₁-C₆, —C(O)C₁-C₆, —C(O)OC₁-C₆, —CF₃, —NO₂, —CH₂O—C₁-C₆, or halo groups.

18. The method of claim 17, wherein said cancer or neoplastic disease is selected from the group consisting of acute leukemia, acute lymphocytic leukemia, acute myelocytic leukemia, myeloblastic, promyelocytic, myelomonocytic, monocytic, erythroleukemia, chronic leukemia, chronic myelocytic (granulocytic) leukemia, chronic lymphocytic leukemia, polycythemia vera, Hodgkin's disease, non-Hodgkin's disease; multiple myeloma, Waldenström's macroglobulinemia, heavy chain disease, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, stadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, and endometrial cancer.

19. The method of claim 17, wherein the cancer cell or neoplastic cell over-expresses an anti-apoptotic Bcl protein.

20. The method of claim 19, wherein the anti-apoptotic Bcl protein is selected from the group consisting of Bcl-2, Bcl-w, Mcl-1, and Bcl-xl.

21. A method for inhibiting the growth of a cancer cell or neoplastic cell comprising contacting the cancer cell or neoplastic cell with an effective amount of a compound or a pharmaceutically acceptable salt thereof having the following features:
(a) a first hydrogen bond donor feature, D1;
(b) a hydrogen bond acceptor feature, A1; and
(c) a second hydrogen bond donor feature, D2;
wherein said D1, A1 and D2 each has a centroid, each centroid being separated by the distances:

| Pair of features | Distance between the features |
|---|---|
| A1-D1 | 2.5-4.5 Å |
| A1-D2 | 2.5-4.5 Å |
| D1-D2 | 3.5-5.5 Å. |

22. The method of claim 21, wherein said cancer cell or neoplastic cell selected from the group consisting of acute leukemia, acute lymphocytic leukemia, acute myelocytic leukemia, myeloblastic, promyelocytic, myelomonocytic, monocytic, erythroleukemia, chronic leukemia, chronic myelocytic (granulocytic) leukemia, chronic lymphocytic leukemia, polycythemia vera, Hodgkin's disease, non-Hodgkin's disease; multiple myeloma, Waldenström's macroglobulinemia, heavy chain disease, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, stadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, and endometrial cancer.

23. The method of claim 21, wherein the cancer cell or neoplastic cell over-expresses an anti-apoptotic Bcl protein.

24. The method of claim 23, wherein the anti-apoptotic Bcl protein is selected from the group consisting of Bcl-2, Bcl-w, Mcl-1, and Bcl-xl.

25. A method for inhibiting the growth of a cancer cell or neoplastic cell comprising contacting the cancer cell or neoplastic cell with an effective amount of a compound or a pharmaceutically acceptable salt thereof having the following features:
(a) a hydrogen bond donor feature, D1;
(b) a hydrogen bond acceptor feature, A1; and
(c) a polar group feature, P1;
wherein said D1, A1, and P1 each has a centroid, each centroid being separated by the distances:

| Pair of features | Distance between the features |
|---|---|
| A1-D1 | 2.5-4.5 Å |
| P1-D1 | 4.5-6.5 Å |
| P1-A1 | 2.5-4.5 Å |

26. The method of claim 25, wherein said cancer or neoplastic cell selected from the group consisting of acute leukemia, acute lymphocytic leukemia, acute myelocytic leukemia, myeloblastic, promyelocytic, myelomonocytic, monocytic, erythroleukemia, chronic leukemia, chronic myelocytic (granulocytic) leukemia, chronic lymphocytic leukemia, polycythemia vera, Hodgkin's disease, non-Hodgkin's disease; multiple myeloma, Waldenström's macroglobulinemia, heavy chain disease, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, stadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, and endometrial cancer.

27. The method of claim 25, wherein the cancer cell or neoplastic cell over-expresses an anti-apoptotic Bcl protein.

28. The method of claim 27, wherein the anti-apoptotic Bcl protein is selected from the group consisting of Bcl-2, Bcl-w, Mcl-1, and Bcl-xl.

29. A method for inhibiting the growth of a cancer cell or neoplastic cell comprising contacting the cancer cell or neoplastic cell with an effective amount of a compound or a pharmaceutically acceptable salt thereof having the following features:
(a) a heterocyclic aromatic ring, Ring A;
(b) a heterocyclic aromatic ring, Ring B, substituted with a polar group;
(c) a heterocyclic aromatic ring, Ring C;
(d) an aliphatic group:
wherein said heterocyclic aromatic ring, Ring A; heterocyclic aromatic ring, Ring B substituted with a polar group; heterocyclic aromatic ring, Ring C; and aliphatic group, each have a centroid, each centroid being separated by the distances:

| | Range of Distances |
|---|---|
| Features | Between Features (Å) |
| heterocyclic aromatic ring (ring A); | 1.5-4.0 |
| heterocyclic aromatic ring (ring B) | |
| substituted with a polar group | |
| heterocyclic aromatic ring (ring B) | 2.5-5 |
| substituted with a polar group; | |
| heterocyclic aromatic ring (ring C) | |
| heterocyclic aromatic ring (ring B) | 4.0-6.5 |
| substituted with a polar group; | |
| aliphatic group | |
| heterocyclic aromatic ring (ring A); | 4.0-6.5 |
| aliphatic group | |
| heterocyclic aromatic ring (ring C); | 3.5-6.5 |
| aliphatic group | |

30. The method of claim 29, wherein said cancer or neoplastic cell selected from the group consisting of acute leukemia, acute lymphocytic leukemia, acute myelocytic leukemia, myeloblastic, promyelocytic, myelomonocytic, monocytic, erythroleukemia, chronic leukemia, chronic myelocytic (granulocytic) leukemia, chronic lymphocytic leukemia, polycythemia vera, Hodgkin's disease, non-Hodgkin's disease; multiple myeloma, Waldenström's macroglobulinemia, heavy chain disease, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, stadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, and endometrial cancer.

31. The method of claim 29, wherein the cancer cell or neoplastic cell over-expresses an anti-apoptotic Bcl protein.

32. The method of claim 31, wherein the anti-apoptotic Bcl protein is selected from the group consisting of Bcl-2, Bcl-w, Mcl-1, and Bcl-xl.

33. A method for inhibiting the growth of a cancer cell or neoplastic cell comprising contacting the cancer cell or neoplastic cell with an effective amount of a compound or a pharmaceutically acceptable salt thereof having the following features:
(a) a first hydrogen bond donor feature, D1;
(b) a hydrogen bond acceptor feature, A1;
(c) a second hydrogen bond donor feature, D2; and
(d) a polar group feature, P1;
said D1, A1, D2 and P1 each has a centroid, each centroid being separated by the distances:

| Pair of features | Distance between the features |
|---|---|
| A1-D1 | 2.5-4.5 Å |
| A1-D2 | 2.5-4.5 Å |
| D1-D2 | 3.5-5.5 Å |
| P1-D1 | 4.5-6.5 Å |
| P1-A1 | 2.5-4.5 Å |
| P1-D2 | 4.5-6.5 Å. |

34. The method of claim 33, wherein said cancer or neoplastic cell selected from the group consisting of acute leukemia, acute lymphocytic leukemia, acute myelocytic leukemia, myeloblastic, promyelocytic, myelomonocytic, monocytic, erythroleukemia, chronic leukemia, chronic myelocytic (granulocytic) leukemia, chronic lymphocytic leukemia, polycythemia vera, Hodgkin's disease, non-Hodgkin's disease; multiple myeloma, Waldenström's macroglobulinemia, heavy chain disease, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, stadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, and endometrial cancer.

35. The method of claim 33 wherein the cancer cell or neoplastic cell over-expresses an anti-apoptotic Bcl protein.

36. The method of claim 35, wherein the anti-apoptotic Bcl protein is selected from the group consisting of Bcl-2, Bcl-w, Mcl-1, and Bcl-xl.

37. A method for inhibiting the growth of a cancer cell or a neoplastic cell comprising contacting the cancer cell or neoplastic cell with an effective amount of a compound of Formula III:
A—B—X—C (III)
or a pharmaceutically acceptable salt thereof,
wherein:
A is selected from the group consisting of 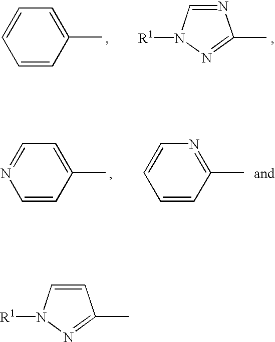
and optionally substituted at one or more carbon atoms with one or more —C₁-C₆, —OC₁-C₆, —OC(O)C₁-C₆, —C(O)C₁-C₆, —C(O)OC₁-C₆, —CF₃, —NO₂, —CH₂O—C₁-C₆, or halo groups;
R¹ is selected from the group consisting of H, —C₁-C₆ and —C(O)C₁-C₆;
B is selected from the group consisting of 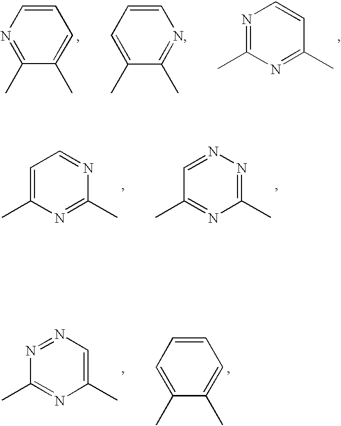
and optionally substituted at one or more carbon atoms with one or more —C₁-C₆, —OC₁-C₆, —OC(O)C₁-C₆, —C(O)C₁-C₆, —C(O)OC₁-C₆, —CF₃, —NO₂, —CH₂O—C₁-C₆, or halo groups,
X is selected from the group consisting of —O—, —S— and —N(H)—; and
C is selected from the group consisting of 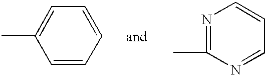
and optionally substituted at one or more carbon atoms with one or more —C₁-C₆, —OC₁-C₆, —OC(O)C₁-C₆, —C(O)C₁-C₆, —C(O)OC₁-C₆, —CF₃, —NO₂, —CH₂O—C₁-C₆, or halo groups.

38. The method of claim 37, wherein said cancer cell or neoplastic cell is selected from the group consisting of acute leukemia, acute lymphocytic leukemia, acute myelocytic leukemia, myeloblastic, promyelocytic, myelomonocytic, monocytic, erythroleukemia, chronic leukemia, chronic myelocytic (granulocytic) leukemia, chronic lymphocytic leukemia, polycythemia vera, Hodgkin's disease, non-Hodgkin's disease; multiple myeloma, Waldenström's macroglobulinemia, heavy chain disease, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, stadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, and endometrial cancer.

39. The method of claim 37, wherein the cancer cell or neoplastic cell over-expresses an anti-apoptotic Bcl protein.

40. The method of claim 39, wherein the anti-apoptotic Bcl protein is selected from the group consisting of Bcl-2, Bcl-w, Mcl-1, and Bcl-xl.
